SEQUENCE LISTING (12 sequences)

SEQ ID NO: 1 - DNA, 25 bp, Artificial sequence
  Synthetic oligonucleotide
cacttaaacccaggaggcagaggtt

SEQ ID NO: 2 - DNA, 24 bp, Artificial sequence
  Synthetic oligonucleotide
accacgtattactggagtcgctga

SEQ ID NO: 3 - DNA, 24 bp, Artificial sequence
  Synthetic oligonucleotide
gataattaaatgttaaaagtcagc

SEQ ID NO: 4 - DNA, 24 bp, Artificial sequence
  Synthetic oligonucleotide
gctgacttttaacatttaattatc

SEQ ID NO: 5 - DNA, 9 bp, Homo sapiens
ggctgagta

SEQ ID NO: 6 - DNA, 8 bp, Artificial sequence
  Synthetic oligonucleotide
gctgagta

SEQ ID NO: 7 - RNA, 22 nt, Artificial sequence
  Synthetic oligonucleotide
aacuaugcaaccuacuaccucu

SEQ ID NO: 8 - DNA, 22 bp, Artificial sequence
  Synthetic oligonucleotide
cttccagtcggggatgtttaca

SEQ ID NO: 9 - DNA, 12 bp, Homo sapiens
gtggctgagtat

SEQ ID NO: 10 - DNA, 12 bp, Mus musculus
ctggctgagtgt

SEQ ID NO: 11 - DNA, 12 bp, Homo sapiens
gtgtatgcctct

SEQ ID NO: 12 - DNA, 12 bp, Mus musculus
gtgtatgcctcc

(19) United States
(12) Patent Application Publication
(10) Pub. No.: US 2012/0045395 A1
(43) Pub. Date: Feb. 23, 2012

(54) MicroRNAs In Idiopathic Pulmonary Fibrosis

(75) Inventors: Naftali Kaminski, Pittsburgh, PA (US); Panagiotis Benos, Pittsburgh (Edgewood), PA (US); David Corcoran, Durham, NC (US); Kusum V. Pandit, Pittsburgh, PA (US); Jadranka Milosevic, Pittsburgh, PA (US); Hanadie Yousef, Berkeley, CA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 13/052,854

(22) Filed: Mar. 21, 2011

Related U.S. Application Data
(63) Continuation of application No. PCT/US2009/057897, filed on Sep. 22, 2009.
(60) Provisional application No. 61/099,298, filed on Sep. 23, 2008.

(51) Int. Cl. A61K 49/00; A61P 11/00; C07H 21/02; C40B 30/04; C12Q 1/68; A61K 31/7088

(52) U.S. Cl. 424/9.1; 435/6.1; 435/6.12; 435/6.11; 514/44 R; 514/44 A; 536/24.5; 506/9

(57) ABSTRACT

The present invention relates to the discovery that certain microRNAs are differentially expressed in Idiopathic Pulmonary Fibrosis. The present invention provides for diagnostic methods, therapeutic methods, and kits related to these differentially expressed microRNAs.

### PRIORITY CLAIM

This application claims priority to U.S. Provisional Application Ser. No. 61/099,298, filed Sep. 23, 2008, the contents of which is hereby incorporated by reference in its entirety.

### GRANT INFORMATION

The subject matter of this patent application was developed at least in part under National Institutes of Health Grant Nos. LM009657, HL073745, and HL0894932, so that the United States Government has certain rights herein.

### 1. INTRODUCTION

The present invention relates to the discovery that certain microRNAs are differentially expressed in Idiopathic Pulmonary Fibrosis. The present invention provides for diagnostic methods, therapeutic methods, and kits related to these differentially expressed microRNAs.

### 2. BACKGROUND OF THE INVENTION

Idiopathic pulmonary fibrosis (IPF) is a chronic, progressive, and usually lethal fibrotic lung disease (Selman M., et al., Annals of internal medicine (2001) 134, 136-151). The disease is characterized by alveolar epithelial cell injury and activation, myofibroblast foci formation, and exaggerated accumulation of extracellular matrix in the lung parenchyma (Selman M., et al., Annals of internal medicine (2001); 134:136-151; American journal of respiratory and critical care medicine (2000); 161:646-664; Katzenstein A L and Myers J L American journal of respiratory and critical care medicine (1998); 157:1301-1315; Gross T J and Hunninghake G W, The New England journal of medicine (2001); 345:517-525). The incidence of IPF in the United States is 16.3 per 100,000 persons (Raghu G., et al. American journal of respiratory and critical care medicine (2006); 174:810-816) with a median survival of 2.5-3 years from diagnosis, and the mortality appears to be increasing (Olson A. L., et al. Am J Respir Crit Care Med (2007); 176:277-284.). The etiology and molecular mechanisms underlying the lung phenotype in IPF are largely unknown.

Recently it was suggested that IPF represents a disease in which developmental pathways are aberrantly activated in the adult (Selman M, et al., PLoS medicine (2008); 5:e62). This suggestion is based on recent evidence for activation of WNT/β-catenin pathway in lung fibrosis (Chilosi M., et al., Am J Pathol (2003); 162:1495-1502; Chilosi M., et al., Respir Res (2006); 7:95; Konigshoff M., et al., PLoS ONE (2008); 3:e2142; Konigshoff M., et al., J Clin Invest (2009); 119:772-787; Vuga L. J. et al., Am J Respir Cell Mol Biol. (2009); 0:2008-0201OCv1) and a potential role for epithelial mesenchymal transition (EMT) in lung fibrosis (Willis B C, et al., The American journal of pathology (2005); 166:1321-1332).

EMT is the phenomenon in which epithelial cells obtain mesenchymal characteristics, including change in shape, increased motility and expression of mesenchymal markers such as N-cadherin (CDH2), vimentin (VIM) and α-smooth muscle actin (ACTA2). EMT is implicated in embryonic development, cancer and kidney fibrosis (Acloque H et al. J Clin Invest (2009); 119:1438-1449; Kalluri R and Weinberg R A, J Clin Invest (2009); 119:1420-1428; Mandal, M., et al., Cancer (2008); 112:2088-2100; Turley E A et al., Nat Clin Pract Oncol (2008); 5, 280-290; Yang, J. and Weinberg, R. A. Developmental cell (2008); 14:818-829; Zeisberg, M. and Kalluri, R., Journal of molecular medicine (Berlin, Germany) (2004); 82:175-181; Iwano M et al. The Journal of clinical investigation (2002); 110, 341-350). TGF-β is considered the major stimulus for EMT through its effects on Snail (SNAI1), Slug (SNAI2), TWIST, ID2 and their regulator HMGA2 (Thuault S. et al., The Journal of cell biology (2006); 174:175-183; Willis, B. C. and Borok, Z., American journal of physiology (2007); 293:L525-534). In-vitro alveolar epithelial cells undergo EMT in response to stimulation by TGF-β and in vivo cells co-expressing epithelial and mesenchymal markers are found in IPF lungs and in mice after intranasal delivery of TGF-β (Willis B. C. et al., The American journal of pathology (2005); 166:1321-1332; Kim K. K. et al., Proceedings of the National Academy of Sciences of the United States of America (2006); 103:13180-13185). In an animal model of lung fibrosis direct evidence that mesenchymal cells were derived from epithelial cells was demonstrated (Kim K. K. et al., Proceedings of the National Academy of Sciences of the United States of America (2006); 103:13180-13185). Kim et al. demonstrated that after intranasal delivery of TGF-13 the majority of lung interstitial myofibroblasts were derived from epithelial cells, suggesting that EMT occurs during lung fibrogenesis and, importantly, that EMT may be more widespread than previously thought (Kim K. K. et al., Proceedings of the National Academy of Sciences of the United States of America (2006); 103:13180-13185).

TGF-β has also been identified as the main inducer of EMT in renal fibrosis (Iwano M. et al., The Journal of clinical investigation (2002); 110:341-350). Iwano et al. have demonstrated that more than a third of the fibroblasts in renal interstitial fibrosis are derived from the renal tubular epithelium (Iwano M. et al., The Journal of clinical investigation (2002); 110:341-350).

Recent studies have suggested a role of microRNAs (also referred as “miRNAs”) in EMT (Burk U. et al., EMBO reports (2008); 9:582-589; Korpal M. et al., The Journal of biological chemistry (2008); 283:14910-14914; Park S. M. et al., Genes & development (2008); 22:894-907; Gregory P. A., et al., Nature cell biology (2008); 10:593-601). MicroRNAs are short, ˜22 nucleotides, and are post-transcriptional gene regulators that function by binding to specific sequences, typically in the 3′ untranslated region of the target mRNAs. Once bound, microRNAs are capable of blocking translation or causing the rapid degradation of the target transcript (Bartel D. P. Cell (2004); 116:281-297). Each microRNA is predicted to have a large number of target genes and each gene is usually predicted to be the target of multiple microRNAs (Krek A. et al., Nature genetics (2005; 37:495-500). MicroRNAs are implicated in embryonic development (Lau N. C. et al., Science (New York, N.Y. (2001); 294:858-862), in multiple cancers including lung cancer (Johnson S. M., et al., Cell (2005); 120:635-647; Wu X., et al., J Thorac Oncol (2009); 4:1028-1034; Nana-Sinkam S. P. et al., American journal of respiratory and critical care medicine (2009); 179:4-10), hepatocellular carcinoma (Murakami Y. et al., Oncogene (2006); 25:2537-2545 (2006)), ovarian cancer (Iorio M. V. et al., Cancer research 2007); 67:8699-8707) and breast cancer (Iorio M. V. et al., Cancer research (2005); 65:7065-7070) and in non-malignant diseases such as chronic heart failure (Thum T. et al., Circulation (2007); 116:258-267; Burk U. et al., EMBO reports (2008); 9:582-589; Korpal M. et al., The Journal of biological chemistry (2008); 283:14910-14914; Park S. M. et al., Genes & development (2008); 22:894-907; Gregory P. A., et al., Nature cell biology (2008); 10:593-601) and are also found in peripheral blood.

### 3. SUMMARY OF THE INVENTION

The present invention relates to diagnostic and therapeutic methods and compositions relating to microRNAs, and is based, at least in part, on the discovery that certain microRNAS are differentially expressed in IPF.

In particular, non-limiting embodiments, the present invention provides for methods of diagnosing IPF in a subject comprising detecting, in a lung tissue sample of the subject, a decrease in the level of one or more microRNA selected from the group consisting of let-7d, mir-30d, mir-30c, mir-30e-5p, miR-26a, miR-26b, miR-29c and mir-17-˜92 cluster and/or an increase in the level of one or more microRNA selected from the group consisting of hsa-mir-154, hsa-mir-205, and hsa-mir31, relative to a control value.

In further particular, non-limiting embodiments, the present invention provides for methods of treating IPF in a subject in need of such treatment comprising administering, to the subject, an agent which increases the level of one or more microRNA selected from the group consisting of let-7d, mir-30d, mir-30c, mir-30e-5p, miR-26, miR-29c and mir-17-˜92 cluster in lung tissue of the subject.

In further particular, non-limiting embodiments, the present invention provides for methods of treating IPF in a subject in need of such treatment comprising administering, to the subject, an agent which decreases the level of one or more microRNA selected from the group consisting of hsa-mir-154, hsa-mir-205, and hsa-mir-31 in lung tissue of the subject.

In still further particular, non-limiting embodiments, the present invention provides for kits to be used in diagnosing IPF comprising means of detecting a change in the level of one or more microRNA selected from the group consisting of let-7d, mir-30d, mir-30c, mir-30e-5p, miR-26a, and miR-26b, miR-29c, mir-17-92, hsa-mir-154, hsa-mir-205, and hsa-mir-31.

### 4. BRIEF DESCRIPTION OF THE DRAWINGS

FIG. 1A-G. MicroRNAs are differentially expressed in IPF. (A) The heatmap on the left represents the global microRNA expression. The heatmap on the right represents statistically significant (p-value <0.05) differentially expressed microRNAs. Up-regulated microRNAs expression levels are shown in progressively brighter shades of yellow, depending on the fold difference, and down-regulated microRNAs are shown in progressively brighter shades of purple. Grey represents that the expression of the microRNAs showed no difference between the two groups being compared. The names of the down-regulated microRNAs are provided to the right of heatmap. (B) A scatterplot representing the microRNAs having q value <0.1 (outlined circles). Let-7d and microRNAs of the miR-26, miR-29 and miR-30 families and the miR-17-92 cluster having q value <0.1 are indicated as dark circles. (C) qRT-PCR verification of the microarray results. The enlarged qRT-PCR verification of the microarray results of reduced let-7d in IPF versus control are shown in D; the enlarged qRT-PCR verification of the microarray results of reduced miR-30c in IPF versus control are shown in E; the enlarged qRT-PCR verification of the microarray results of reduced miR-30d in IPF versus control are shown in F; and the enlarged qRT-PCR verification of the microarray results of reduced miR-30e-5p in IPF versus control are shown in G.

FIG. 2A-D. Let-7d is a TGF-β target molecule. (A) Putative SMAD3 binding sites identified by the FOOTER algorithm upstream of hsa-let-7d microRNA identified to be differentially expressed in IPF versus control lung. “HS” represents human sequence, and “MM” represents mouse sequence. (B) A549 cells were treated with 3 ng/ml recombinant TGF-β and let-7d expression was determined at 0 hour, 2 hours and 6 hours post-stimulation. The results represented an average expression±S.D. of triplicate experiments. (C) Putative SMAD3 binding sites identified by the FOOTER algorithm upstream of miR-30c-2 microRNA. “HS” represents human sequence, and “MM” represents mouse sequence. (D) A549 cells were treated with 10 ng/ml recombinant TGF-β and miR-30c expression was determined at 0 hour, 2 hours and 6 hours post-stimulation. The results represented an average expression of triplicate experiments.

FIG. 3A-E. The putative promoter of let-7d was bound by SMAD3 and responsive to TGF-β. (A) Electrophoretic mobility shift assay and (B) supershift assays of recombinant SMAD3 protein and nuclear extracts isolated from A549 cells treated with 2 ng/ml recombinant human TGF-β for 1 hour. (C) SMAD3 ChIP assay revealed association with let-7d in A549 lung cells. (D) Reporter assays were performed on A549 cells transfected with a recombinant vector containing −1600 to +100 base pair let-7d region 5′ to luciferase gene. The luciferase scale is arbitrary and values are average of the triplicate assays. TGF-β stimulation was carried out before DNA transfection. The dark blue bars represent the reporter construct and the light blue bars represent the empty vector. (E) Reporter assays were performed on A549 cells transfected with a recombinant vector containing −1600 to +87 base pair let-7d region 5′ to luciferase gene referred to as p-let-7d-luc. A similar vector with an 8 bp deletion of the predicted SMAD3 binding site is referred to as p-mlet-7d-luc. The luciferase scale is arbitrary and values are average of the triplicate assays. TGF-β stimulation was carried out before DNA transfection. The dark blue bars represent the reporter construct without any stimulation and the light blue bars represent the same with TGF-β stimulation.

FIG. 4A-J. Inhibition of let-7d results in EMT. (A) HMGA2 mRNA levels determined by qRT-PCR in A549 cells at 0 hour, 2 hours and 6 hours post-stimulation with 3 ng/ml recombinant TGF-β. The results represent an average expression±S.D. of triplicate experiments. (B) HMGA2 mRNA levels determined by qRT-PCR in A549 cells at 24 hours and 48 hours post-transfection with 50 nM of let-7d inhibitor. (C) HMGA2 mRNA levels in RLE-6TN cells at 0 hour, 2 hours and 6 hours post-stimulation with 5 ng/ml recombinant TGF-β following transfection with pre-let-7d or negative control 24 hours earlier. CDH2 (N-cadherin), VIM (vimentin) and ACTA2 (alpha smooth muscle actin) mRNA levels determined by qRT-PCR in A549 cells (D) and RLE-6TN cells (E) at 48 h post-transfection and (F) NHBE cells at 24 hours post-transfection with 50 nM of let-7d inhibitor. (G-J) Immunofluorescence imaging of A549 cells transfected with 50 nM of let-7d inhibitor. The green fluorescence represented cytokeratin, an epithelial marker. The red fluorescence denotes the mesenchymal markers (CDH2 (N-Cadherin), VIM (Vimentin), (ACTA2 (α-smooth muscle actin)). Nuclei were counterstained with DAPI. While red staining is observed in cells transfected with let-7d inhibitor (right panel), there is no staining in cells transfected with a control oligonucleotide (left panel).

FIG. 5A-C. Tissue microarray analysis reveals that let-7d localized within normal alveolar epithelium in control lungs and was nearly absent from fibrotic areas in IPF lungs. (A) Panels i and iii: let-7d was localized in alveolar epithelial cells of control lungs as evident by the black staining (blue arrows). Panels ii and iv: IPF lungs showed almost a total absence of let-7d. The black arrows point to areas of dense fibrosis while blue arrows point to minimal staining for let-7d in the immediate surrounding areas. Scale bars in panels i and ii denote 100 μm while those in iii and iv represent 25 μm. (B) Number of let-7d expressing cells/mm³ was significantly lower in 40 slides from patients with IPF compared to 20 normal histology controls. (C) The FVC of IPF patients significantly increased (p value=2.1E-6) with the number of let-7d expressing cells/mm³.

FIG. 6A-E. HMGA2 localizes to alveolar epithelial cells. (A) HMGA2 mRNA levels determined by qRT-PCR in 10 control and 10 IPF lungs. (B-E) Immunolocalization of HMGA2 in IPF (B-D) and normal lungs (E). Panels (B) and (C): Two different IPF lungs showing the immunoreactive protein was found primarily in alveolar epithelial cells, either in cytoplasm or nuclei (black arrows). The red arrows in (B) point to collapsed air spaces which are lined by HMGA2 positive epithelial cells. Panel (D): The same IPF lungs from C showing nuclei staining of HMGA2 in elongated epithelial cells (asterisk indicates an alveolar space) and some fibroblast-like cells immersed in a fibroblastic focus. Positive endothelial cells are marked with red arrows. Panel (E): Normal lungs were negative for HMGA2. The scale bars in all B-E denote 100 μm length.

FIG. 7A-F. Effect of let-7 inhibition in vivo by intratracheal antagomir administration. Gene expression levels of (A) CDH1 (B) ZO-1, (C) COL1A and (D) HMGA2 levels in mice (n=4) treated with 10 mg/kg intratracheal antagomir or saline for 4 days. Panels (i) and (iii) in (E) and (F), are saline lungs and panels (ii) and (iv) in (E) and (F) are antagomir-treated lungs. The scale bars in (i) and (iii) in (E) and (F) denote 100 μm and those in (ii) and (iv) in (E) and (F) denote 25 μm. (E) Masson trichrome staining after 18 days of saline or antagomir treatment. Arrows in (ii) and (iv) in (E) and (F) point to the blue staining for collagen. Note alveolar septal thickening. (F) Immunolocalization of α-smooth muscle actin in Group III mice. ACTA2 staining (brown) is significantly increased in antagomir-treated mice compared to saline treated controls.

FIG. 8A-B. Down-regulated microRNAs have many overlapping targets. (A) Representation of the computationally predicted targets of a few down-regulated microRNAs color-coded by the number of sites in the 3′UTR as predicted by TargetScan. (B) Direct comparison of microarray gene expression between IPF and control. X axis—Average gene expression in control, Y axis—Average gene expression in IPF. Red circles represent the genes in (A) and significantly different between IPF and control lungs.

FIG. 9A-D. Specificity of the let-7d inhibitor and effect of HMGA2 inhibition on expression of mesenchymal markers. Expression levels of (A) let-7d (B) let-7c and (C) miR-10a determined by qRT-PCR after transfecting A549 cells with a let-7d inhibitor. (D) Inhibition of HMGA2 did not completely ablate increase in CDH2, ACTA2 and VIM determined by qRT-PCR in A549 cells after 48 hours of let-7d inhibition alone and in combination with HMGA2 inhibition. “Scr mir” is the negative control for let-7d inhibition and “scr scr” refers to the negative controls for let-7d inhibition and HMGA2 inhibition.

FIG. 10. Efficacy of the let-7d antagomir. Expression levels of let-7d in mice lungs after 10 mg/kg antagomir treatment for 4 days and 5 mg/kg antagomir treatment for 18 days.

FIG. 11A-C. Results of real-time PCR studies of levels of (A) hsa-miR-30c; (B) hsa-miR-30d; and (C) hsa-miR-30e-5p in IPF lung tissue as compared to control lung tissue.

FIG. 12A-D. In situ hybridization studies using a miR-30e probe on sections of lung tissue from (A) control lung (40×); (B) IPF lung (40×); (C) control lung (40×); and (D) IPF lung (20×), where hybridization is indicated by red staining.

FIG. 13. Result of real-time PCR analysis of the levels of NFYC in tissue from control and IPF lung tissue.

FIG. 14A-C. Results of real-time PCR experiments to determine levels of microRNAs in A549 lung alveolar cells transfected with an LNA/DNA test oligonucleotide (5′ CTTCCAGTCGGGGATGTTTACA 3′ (SEQ ID NO: 8), where underlined bases are LNA bases and the rest of them are DNA bases, see Mott J. L. et al., Oncogene (2007);26(42):6133-40). MicroRNAs evaluated, at 24 and 48 hours post-transfection, were (A) miR-30a-5p; (B) miR-30d; and (C) miR-30e-5p.

FIG. 15A-D. Results of real-time PCR experiments to determine levels of various mRNAs in the transfected cells of FIG. 14A-C, where the mRNAs evaluated, at 24 and 48 hours post-transfection, were (A) HMGA2; (B) vimentin; (C) ADAM 19 and (D) EDNRA.

FIG. 16. Diagram of proposed relationship between miR-30 microRNAs and the expression of various genes associated with IPF.

FIG. 17. Result of real-time PCR analysis of the levels of hsa-mir-154 in tissue from control and IPF lung tissue.

FIG. 18. Result of real-time PCR analysis of the levels of hsa-mir-205 in tissue from control and IPF lung tissue.

FIG. 19. Result of real-time PCR analysis of the levels of hsa-mir-31 in tissue from control and IPF lung tissue.

FIG. 20. Result of real-time PCR analysis of the levels of hsa-mir-324-3p in tissue from control and IPF lung tissue.

FIG. 21. Result of real-time PCR analysis of the levels of hsa-mir-155 in tissue from control and IPF lung tissue.

FIG. 22. Effect of antagomir on let-7d expression in vivo.

### 5. DETAILED DESCRIPTION OF THE INVENTION

For clarity of description and not by way of limitation, the detailed description of the invention is divided into the following subsections:

(i) diagnostic methods;

(ii) therapeutic methods; and

(iii) kits.

### 5.1 Diagnostic Methods

In particular, non-limiting embodiments, the present invention provides for methods of diagnosing IPF in a subject comprising measuring, in a lung tissue sample of the subject, the level of one or more microRNA, wherein a decrease in the level of one or more microRNA selected from the group consisting of let-7d, mir-30d, mir-30c, mir-30e-5p, miR-26a, miR-26b, miR-29c and mir-17-˜92˜ cluster and/or an increase in the level of one or more microRNA selected from the group consisting of hsa-mir-154, hsa-mir-205, and hsa-mir-31, relative to one or more control value, indicates a diagnosis of IPF in the subject. “miR-26” used herein, includes, but is not limited to miR-26a and miR-26b. Said method may further comprise administering one or more pulmonary function test, such as, but not limited to, diffusing capacity of carbon monoxide, vital capacity, total lung capacity, forced vital capacity and/or high resolution computer assisted tomography in order to confirm (or refute) the diagnosis of IPF. The method may further comprise a recommendation relating to therapy options, including but not limited to lung transplant.

The level of microRNA may be measured by any method known in the art, including but not limited to hybridization-based methods, for example Northern blotting or chip-based methods (e.g Agilent Human microRNA Microarray) as well as polymerase-based methods, such as quantitative real-time polymerase chain reaction (“qRT-PCR”).

The lung tissue sample may be obtained by biopsy, by bronchoalveolar lavage, or by any analogous method, and as such, may comprise individual cells.

The control value may be obtained by assaying lung tissue from a normal control subject to generate a basis for comparison (either in a parallel experiment or by prior generation of a standard value) or may be a published value.

An “increase” as that term is used herein means an increase of at least about 25% or of at least about 50% relative to a control value or to the mean of a plurality of normal values.

A “decrease” as that term is used herein means a decrease of at least about 25% or of at least about 50% relative to a control value or to the mean of a plurality of normal values.

### 5.2 Therapeutic Methods

In particular, non-limiting embodiments, the present invention provides for methods of treating IPF in a subject in need of such treatment comprising administering, to the subject, an agent which increases the level of one or more microRNA selected from the group consisting of let-7d, mir-30d, mir-30c, mir-30e-5p, miR-26a, miR-26b, miR-29c and mir-17-˜92 cluster in lung tissue of the subject. miR-26 family includes, but is not limited to, miR-26a and miR-26b.

In certain specific embodiments, the agent that increases the level of said one or more microRNAs may be a nucleic acid encoding said microRNA in expressible form. For example, said nucleic acid may comprise a sequence encoding said microRNA operably linked to a suitable promoter. A suitable promoter may be selectively or constitutively active in a lung cell, such as an epithelial alveolar cell. Specific non-limiting examples of suitable promoters include constitutively active promoters such as the cytomegalovirus immediate early gene promoter, the Rous sarcoma virus long terminal repeat promoter, the human elongation factor 1α promoter, and the human ubiquitin c promoter. Specific non-limiting examples of lung-specific promoters include the surfactant protein C gene promoter, the surfactant protein B gene promoter, and the Clara cell 10 kD (“CC 10”) promoter. Said nucleic acid may optionally be comprised in a vector, which may be a viral or non-viral vector. Non-limiting examples of viral vectors that infect lung cells include, but are not limited to, adenovirus vectors, adeno-associated virus vectors, and paramyxovirus vectors.

In further particular, non-limiting embodiments, the present invention provides for methods of treating IPF in a subject in need of such treatment comprising administering, to the subject, an agent which decreases the level of one or more microRNA selected from the group consisting of hsa-mir154, hsa-mir205, and hsa-mir31, in lung tissue of the subject.

In certain specific embodiments, the agent that decreases the level of said one or more microRNAs may be a nucleic acid encoding an antisense RNA or interfering RNA complementary, at least in part, to said microRNA, in expressible form (where the antisense RNA “targets” the microRNA). Complementary at least in part means that the antisense RNA is at least about 90 percent or at least about 95 percent homologous to the microRNA, where homology is determined by standard software such as BLAST or FASTA. For example, said nucleic acid may comprise a sequence encoding an antisense RNA which targets the microRNA of interest, operably linked to a suitable promoter. A suitable promoter may be selectively or constitutively active in a lung cell, such as an epithelial alveolar cell. Specific non-limiting examples of suitable promoters include constitutively active promoters such as the cytomegalovirus immediate early gene promoter, the Rous sarcoma virus long terminal repeat promoter, the human elongation factor 1α promoter, and the human ubiquitin c promoter. Specific non-limiting examples of lung-specific promoters include the surfactant protein C gene promoter, the surfactant protein B gene promoter, and the Clara cell 10 kD (“CC10”) promoter. Said nucleic acid may optionally be comprised in a vector, which may be a viral or non-viral vector. Non-limiting examples of viral vectors that infect lung cells include, but are not limited to, adenovirus vectors, adeno-associated virus vectors, and paramyxovirus vectors.

A specific, non-limiting example of a nucleic acid which may be used to decrease levels of let-7d microRNA is the “Antagomir” oligonucleotide (5′ aacuaugcaaccuacuaccucu 3′ (SEQ ID NO: 7), where all bases are 2′-OMe-modified, the first two and last four bases have phosphorothioate linkages, and a cholesterol molecule is attached at the 3′ end). The present invention provides for therapeutic compositions comprising Antagomir or other antisense or interfering RNA molecules that inhibit the activity of target microRNAs described herein, in a suitable pharmaceutical carrier.

An agent which either increases or decreases target microRNA levels may be administered to a subject systemically and/or directly into the lungs, for example by inhalation or lavage.

### 5.3 Kits

In still further particular, non-limiting embodiments, the present invention provides for kits to be used in diagnosing IPF comprising a means for detecting a change in the level of one or more IPF-associated microRNA selected from the group consisting of let-7d, mir-30d, mir-30c, mir-30e-5p, miR-26a, miR-26b, miR-29c, mir-17-˜92 cluster, hsa-mir-154, hsa-mir-205, and hsa-mir-31. miR-26 family includes, but is not limited to, miR-26a and miR-26b.

A means for detecting a change in the level of an IPF-associated microRNA may comprise a nucleic acid comprising a sequence complementary to said microRNA (a “detector nucleic acid”). In one set of non-limiting embodiments, the detector nucleic acid may be bound to a solid substrate, for example as a “dot blot” or as part of an array representing a plurality of RNAs. In another set of non-limiting embodiments, the detector nucleic acid may be used in a qRT-PCR reaction to determine the level of microRNA. Preferably, but not by way of limitation, the nucleic acid is a DNA, but it may also be a ribonucleotide or a nucleic acid containing synthetic/modified nucleotide analogs; a specific example is a nucleic acid comprising one or more methylated nucleic acid and/or one or more phosphorothioate linkage.

Said kit may further contain a detector nucleic acid to be used to generate a control value representing an RNA having a level which is not expected to change in IPF versus healthy lung. Non-limiting examples of such RNAs include hsa-mir324-3p, and hsa-mir155.

In non-limiting examples of the invention, the one or more IPF-associated microRNA(s) specie(s) constitute at least about 10 percent, at least about 20 percent, at least about 30 percent, at least about 40 percent, at least about 50 percent, at least about 60 percent, at least about 70 percent, at least about 80 percent, or at least about 90 percent, of the total number of RNA species represented in (testable by, measurable by) the kit.

### 6. EXAMPLES

### 6.1 Example 1

### Inhibition and Role of Let-7d in IPF

6.1.1 Materials and Methods

IPF tissues:—10 IPF Lung tissue samples and 10 controls for microarray analysis were obtained through the University of Pittsburgh Health Sciences Tissue Bank as described in Rosas I. O. et al., PLoS medicine (2008); 5:e93; Pardo A. et al., PLoS medicine (2005); 2:e251. 10 samples were obtained from surgical remnants of biopsies or lungs explanted from patients with IPF who underwent pulmonary transplant and 10 control normal lung tissues obtained from the disease free margins with normal histology of lung cancer resection specimens. The morphologic diagnosis of IPF was based on typical microscopic findings consistent with usual interstitial pneumonia (Katzenstein A. L. and Myers J. L. American journal of respiratory and critical care medicine (1998); 157:1301-1315). All patients fulfilled the diagnostic criteria for IPF outlined by the American Thoracic Society and European Respiratory Society (American journal of respiratory and critical care medicine (2000); 161:646-664). All studies were approved by the Institutional Review Board at the University of Pittsburgh.

Cell culture: A549 cells (CCL-185, American Type Culture Collection (ATCC), Manassas, Va.) and RLE-6TN cells (CRL-2300, ATCC) were grown in F12K medium (Invitrogen, Carlsbad, Calif.) with 2 mM L-glutamine and 10% fetal bovine serum at 37° C. in a humidified chamber supplemented with 5% CO₂. Wherever indicated cells were stimulated with TGF-β (R&D, Minneapolis, Minn.).

RNA Isolation: Total RNA from tissues and A549 cells was isolated using the miRNeasy Mini kit (Qiagen, Valencia, Calif.) according to the manufacturer's instructions. The quantity of the RNA was determined by optical density, measured at 260 nm by Nanodrop spectrophotometer. RNA quality was measured using the RNA 6000 Nano kit and the small RNA kit on the Agilent Bioanalyzer 2100.

miRNA Microarray: Total RNA from tissues and cells was isolated using the miRNeasy Mini kit (Qiagen, Valencia, Calif.). MicroRNA profiling was carried out on the Agilent Human miRNA Microarray using the manufacturer's protocol. These microarrays have an 8×15K design with 470 microRNAs based on Release 9.1 of Sanger miRBASE. The manufacturer's instructions were followed in the labeling and hybridization of the RNA. The gene expression microarrays were described in Konishi K. et al., American journal of respiratory and critical care medicine (2009); 180:167-175. 100 ng of total RNA was dephosphorylated using calf intestine alkaline phosphatase (GE Healthcare, Piscataway, N.J.), denatured with DMSO, and labeled with pCp-Cy3 using T4 RNA ligase (New England Biolabs, Ipswich, Mass.) at 16° C. for 2 hours. The labeled RNA was purified using Micro Bio-spin 6 columns and hybridized onto the Agilent miRNA microarrays at 55° C. for 20 hours. The arrays were washed with Gene Expression Wash Buffers 1 and 2 (Agilent) and scanned using the Agilent Microarray Scanner. The scanned images were processed by Agilent's Feature Extraction software version 9.5.3.

Statistical analysis: MicroRNA microarray data was log 2 transformed, normalized to the mean of each array and a Wilcoxon rank-sum test was used to identify those microRNAs that were differentially expressed (p-value <0.05) between IPF and control lungs; each microRNA has 3-4 unique probes on the array. Only microRNAs whose mean values for each probes had an expression value >95% of the negative controls in at least one condition were considered for statistical analysis. Data visualization was accomplished using Genomica (http://genomica.weizmann.ac.il) (Segal E. Nature genetics (2004); 36:1090-1098) and Spotfire Decision Site 8.0 (Spotfire Inc., Göteborg, Sweden, http://spotfire.tibco.com). For qRT-PCR, statistical significance was determined by Student's t-test using p<0.05. In situ hybridizations were analyzed by student t-test and Mann-Whitney test to compare let-7d positive AECs/mm² between IPF and control lung samples.

Quantitative RT-PCR: TaqMan MicroRNA assays (ABI, Foster City, Calif.) were used to determine the relative expression levels of microRNAs, e.g., hsa-let-7d, miR-30c, miR-30d and miR-30e-5p. For RT reactions, 50 ng of total RNA was used to determine the relative expression levels of microRNAs and mRNAs respectively.

The quantity of the RNA was determined by optical density, measured at 260 nm by Nanodrop spectrophotometer. RNA quality was measured using the RNA 6000 Nano kit and the small RNA kit on the Agilent Bioanalyzer 2100. For RT reactions, 50 ng of total RNA was used in each 15 μl reaction. The conditions for the RT reaction were: 16° C. for 30 minutes; 42° C. for 30 minutes; 85° C. for 5 min; and then held on 4° C. The cDNA was diluted 1:14 and 1.33 μl of the diluted cDNA was used with the TaqMan primers in the PCR reaction. The conditions for the PCR were: 95° C. for 10 minutes followed by 40 cycles of 95° C. for 15 seconds and 60° C. for 1 minute in the ABI 7300 real-time PCR system. The results were analyzed by the ΔΔCt method using RNU43 control RNA for normalizing human microRNAs and snoRNA55 for mouse microRNAs. Fold change was calculated taking the mean of the controls as the baseline. TaqMan gene expression assays (ABI) were used to determine the relative expression levels of HMGA2, CDH2, VIM, ACTA2, CDH1, ZO1 and COL1A. 500 ng of RNA was reverse transcribed using the SuperScript First-Strand Synthesis System for RT-PCR (Invitrogen) in a total reaction volume of 20 μl, the cDNA diluted 1:5 and 3 μl of this cDNA was used in a total volume of 31 μl for the PCR. PCR conditions were as follows: 12 minutes at 95° C., followed by 40 cycles with 15 seconds at 95° C. and 1 minute at 60° C. in the ABI 7300 real-time PCR system. The results were analyzed by the ΔΔCt method and GUSB was used for normalization. Fold change was calculated taking the mean of the controls as the baseline.

miRNA promoter analysis: Genomic coordinates of all differentially expressed microRNAs between IPF and control lungs and the coordinates of their murine orthologues were obtained from the UCSC Genome Browser (Kent W. J. et al., Genome research (2002); 12:996-1006). The 1 kb sequence upstream of the intergenic microRNAs and the 1 kb sequence upstream of the host gene for the intronic microRNAs were collected. The host gene promoter sequence was used for intronic microRNAs because previous reports have shown that the microRNA and host gene are co-transcribed and share the same promoter region (Baskerville S, and Bartel D. P., RNA (New York, N.Y.) (2005); 11:241-247). SMAD3 and SMAD4 binding site prediction was carried out with the FOOTER algorithm using default parameters (Corcoran, D. L., et al., Genome research (2005); 15:840-847).

Chromatin Immunoprecipitation (ChIP): The ChIP protocol was performed according to the published protocol from the Young laboratory (Lee T. I. et al., Science (New York, N.Y.) (2002); 298:799-804). A549 cells were grown to 5×10⁷-1×10⁸ cells per analysis condition. Cells were either untreated (control) or stimulated with 2 ng/mL TGF-β for 30 minutes. Chromatin cross-linking was performed by adding 1/10 volume of freshly prepared 11% formaldehyde solution for 15 minutes at room temperature. The cross-linking reaction was then quenched by adding 1/20 volume of 2.5M glycine. Cells were rinsed twice with PBS, collected with a silicon scraper, flash frozen in liquid nitrogen, and stored at −80° C. until use. Upon thawing, cells were resuspended in a lysis buffer and sonicated at 4° C. to solubilize cellular components and shear crosslinked chromatin. The cell lysate was incubated overnight at 4° C. with 100 μA of Dynal Protein G magnetic beads that had been preincubated with 10 μg of either anti-flag (mock IP) or anti-SMAD3 antibodies (Millipore, Billerica, Mass.). Protein G magnetic beads were washed five times with RIPA buffer and one time with TE buffer containing 50 mM NaCl. Cross-linked promoter fragment/transcription factor complexes were eluted from the beads by heating at 65° C. with vortexing at 2 minute intervals for 15 minutes. Crosslinking was reversed by incubation at 65° C. overnight. Recovered promoter fragments were treated with RNaseA, proteinase K digestion, and purified by phenol:chloroform:isoamyl alcohol extraction/ethanol precipitation. Gene-specific PCR was performed on a portion of the purified recovered nucleic acid (25 cycles) to verify the presence of the upstream sequence of pre-hsa-let-7d. The primers used for gene-specific PCR are: let-7d forward: 5′-CAC TTA AAC CCA GGA GGC AGA GGT T-3′ (SEQ ID NO: 1) and let-7d reverse: 5′-ACC ACG TAT TAC TGG AGT CGC TGA-3′ (SEQ ID NO: 2).

Electrophoretic Mobility Shift Assay (EMSA): Cultured A549 cells at 60-70% confluence were treated with 2 ng/mL recombinant human TGFβ₁ (R&D Systems) for 60 minutes. Nuclear proteins were isolated using a standard rapid micropreparation technique described at Andrews N. C. and Faller D. V. Nucleic acids research (1991); 19:2499). The supernatant was reserved and snap frozen in liquid nitrogen as the nuclear protein fraction. Nuclear extracts and recombinant full length SMAD3 protein (Santa Cruz Biotechnology, Santa Cruz, Calif.) were incubated with 5′-end Cyanine-5 labeled probe and/or non-labeled competitor oligonucleotide for 20 minutes at room temperature in a binding buffer consisting of 20% glycerol, 5 mM MgCl₂, 2.5 mM EDTA, 25 mM DTT, 200 mM NaCl, 50 mM Tris HCl pH 7.6, and 0.25 mg/mL poly(dI-dC). The complementary oligonucleotides (5′-GATAATTAAATGTTAAAAGTCAGC-3′ (SEQ ID NO: 3), 5′-GCTGACTTTTAACATTTAATTATC-3′ (SEQ ID NO: 4)) were synthesized by Integrated DNA Technologies (Coralville, Iowa), and consisted of a sequence upstream of the predicted SMAD3/let7d binding site (GGCTGAGTA (SEQ ID NO:5)). Additionally, a supershift assay was performed by incubating nuclear extract with 0.1 μl rabbit monoclonal antibody [EP568Y] to SMAD3 (Abcam, Cambridge, Mass.) or 1.0 μl mouse monoclonal [4A3] to peroxiredoxin 6 as a control (Abcam) prior to incubating with the target oligonucleotide. The protein/DNA complexes were run on a 6% native polyacrylamide gel and visualized on a Typhoon imaging and documentation system using Cyanine-5 dye excitation and fluorescence settings.

Luciferase reporter assays: pGL4.17 (Promega, Madisson, Wis.) constructs contained −1600 to +87 base pair region of let-7d at the 5′ end of the reporter gene or the 8 base pair Smad3 site deletion (GCTGAGTA (SEQ ID NO: 6)) plasmid constructed using Quick Change mutagenesis kit (Stratagene, La Jolla, Calif.). 80% confluent A549 cells were stimulated for 2 hours with 10 ng/ml TGF-β. Reporter DNA was transfected using Lipofectamine 2000 (Invitrogen) at 1:1 ratio for 4 hours and cell growth continued for another 16 hours. Luciferase activity was determined using dual-reporter assay system (Promega).

Transfection: A549 cells and RLE-6TN cells were transfected with 50 nM hsa-let-7d inhibitor, pre-let-7d and their corresponding negative controls (Ambion, Austin, Tex.) using Lipofectamine 2000 (Invitrogen) according to the manufacturer's instructions. RNA was isolated 24 hours and 48 hours post-transfection.

Immunofluorescence: A549 cells were transfected with 50 nM anti-let7d for 48 hours and stained for cytokeratin, VIM, CDH2 and ACTA2. A549 cells were plated on cover slips. Cells were starved for 24 hours by the removal of serum and transfected with 50 nM anti-let7d for 48 hours. Cover slips were removed, and fixed in 2% paraformaldehyde (Sigma) for 40 minutes. Permeabilization of cells was carried out by using 0.1% Triton X in PBS for 40 minutes, with three washes in PBS each for five minutes followed by blocking with 0.3% BSA and 5% goat serum in PBS for 60 minutes, and incubated with anti-cytokeratin, anti-vimentin, anti-N-Cadherin, or anti-alpha-smooth muscle actin (all from Abcam Inc., Cambridge, Mass.) in 0.5% BSA in PBS for 60 minutes. Following three washes with 0.5% BSA in PBS (5 minutes each), coverslips were incubated with the appropriate secondary antibody (Invitrogen, Carlsbad, Calif.) for one hour at 37° C. After staining, cover slips were washed with 0.1% Triton in PBS for 2 times 5 minutes each followed by three washes with PBS 5 minutes each. Coverslips were inverted onto slides and mounted in Vectashield anti-fade medium that contained DAPI for nuclei staining (Vector Laboratories, Burlingame, Calif.) to prevent photobleaching. Slides were examined using a Leica TCS-SP2 laser scanning confocal microscope equipped with appropriate lasers for simultaneous imaging of up to four fluorophores. Digital data was archived to compact disk or DVD and prepared for publication using Adobe Photoshop software (Adobe Systems Inc., MountainView, Calif.).

Tissue microarray construction: Tissues were snap-frozen and stored at −70° C. Specimens were fixed in cold-ethanol for 16 hours and then embedded in paraffin. Hematoxylin and eosin (H&E)-stained sections were made from each block to define representative fibrotic and inflammatory lesion regions. Areas of interest were identified in H&E stained slides by a conventional microscope (Olympus BX-50). Tissue cylinders with a diameter of 1.5 mm were punched from selected areas of each “donor” block using a thin-wall stainless tube from a precision instrument (TMA-100, Chemicon, USA) and were transferred by a solid stainless stylet into defined array coordinates in a 45*20 mm new recipient paraffin block. The tissue microarray blocks were constructed in three copies (each containing one sample from a different region of all lesions). One sample was taken from the center and two samples from different peripheral areas. Ultimately, we constructed three tissue microarray blocks comprising of 80 tissue elements each. Each tissue element in the array was 1.5 mm in diameter and spacing between two adjacent elements was 0.1 mm. After the TMA construction 5 μm sections for in situ hybridization analysis were cut from the “donor” blocks and were transferred to glass slides using an adhesive-coated tap sectioning system.

In situ hybridization: The tissue microarrays were described in Tzouvelekis A. et al., Am J Respir Crit Care Med (2007); 176:1108-1119. A total of 60 tissue samples consisting of 40 IPF and 20 control tissues derived from the normal part of lungs removed from benign lesions were studied. The number of let-7d positive alveolar epithelial cells (AECs)/mm² in 5 fields per case was counted. Lung tissues were obtained from the tissue bank of two different pathology centers (University Hospital of Alexandroupolis, Greece and the Veterans Administration Hospital, N.M.T.S., Athens, Greece). The tissues were fixed in 10% formalin, paraffin-embedded and after the TMA construction samples were cut into 5 μm thick serial sections and were transfected to glass slides using an adhesive coated tap sectioning system. The paraffin sections were dewaxed in xylene for 2×5 minutes, soaked in 100% ethanol. Then the paraffin sections were soaked in 75% ethanol and after in wash buffer solution so the tissue can retain its initial pH. The sections were then treated with proteolytic solution supplied in the kit for 30 min at 37° C. Excess proteolytic solution was discarded and the slides were dehydrated in 75%, 95% and 100% ethanol for 1 minute each and then air-dried. Denaturation and hybridization were done for overnight at 37° C. with 20 nM 5′-digoxigenin-labeled miRCURY LNA detection probe (Exiqon, Denmark) diluted in hybridization buffer (50% Formamide, 5×SSC, 0.1% Tween, 9.2 mM citric acid for adjustment to pH6, 50 ug/ml heparin, 500 ug/ml yeast RNA). The slides were washed in TBS buffer for 3×1 minute. Slides were transferred onto a 37° C. heating block or slide warmer and 2-3 drops of alkaline-phosphatase conjugate were applied to the specimen. Slides were then incubated for 30 min at 37° C. Excess detection reagent was tapped off and slides were washed in TBS buffer. Slides were then soaked in three changes of TBS buffer for 1 minute each and then transferred into a container with distilled or deionized water and soaked for 1 additional minute. They were then taken out; excess of water was wiped off and dried around the edges using a lint-free cloth. Sections were then transferred onto a 37° C. heating block and 2-3 drops of NBT/BCIP substrate were applied to each specimen. Then slides were incubated in the dark for 5-15 minutes at 37° C. (color development was examined every 5 minutes with a light microscope) and removed from the heating block. They were then washed three times for 1 minute in changes of distilled or deionized water. The slides were counterstained using Nuclear Fast Red. 2-3 drops of counterstain were applied to each slide (with hematoxylin for 10 sec) and rinsed in distilled water for 3 min. Slides were then incubated for 15 sec, excess of counterstain was tapped off and then they were washed briefly in distilled or deionized water. Images were acquired by using the high-resolution DUET, BioView scanning system for CISH, morphology applications.

CISH semi-quantitative image analysis: The number of let-7d positive alveolar epithelial cells (AECs)/mm² in 5 fields per cases was counted by two independent pathologists—observers using the high resolution DUET, BioView scanning system for CISH morphology and immunocytochemistry applications, at ×100 magnification. Independent t-test and Mann-Whitney test were used to compare let-7d positive (AECs)/mm² between IPF and control lung samples.

Immunohistochemistry: Tissue sections were treated as described at Selman M. et al., American journal of physiology (2000); 279:L562-574. Anti-HMGA2 rabbit polyclonal antibody (4 μg/ml) (Abcam) and anti-ACTA2 (ab21027, Abcam) were used. Anti-HMGA2 rabbit polyclonal antibody (4 μg/ml) (Abcam) was applied and samples were incubated at 4° C. overnight. A secondary biotinylated anti-immunoglobulin followed by horseradish peroxidase-conjugated streptavidin (BioGenex, San Ramon, Calif.) was used according to manufacturer's instructions. 3-amino-9-ethyl-carbazole (BioGenex) in acetate buffer containing 0.05% H₂O₂ was used as substrate. The sections were counterstained with hematoxylin. The primary antibody was replaced by non-immune serum for negative control slides. Mouse lung tissue was fixed and inflated with PROTOCOL SafeFix (Fisher Scientific, Waltham, Mass.). Tissue sections were deparaffinized and rehydrated using xylene and sequential ethanol rinses, endogenous peroxidase activity blocked with methanol and hydrogen peroxide, antigen retrieval done by heating the slides in sodium citrate buffer pH 6.0 at 95° C. for 30 minutes followed by blocking in 5% goat serum for 30 minutes. The sections were incubated with an antibody to ACTA2 (ab21027, Abcam) for 1 hour at room temperature. Following two 5 minutes washes in TBS, the sections were incubated in the secondary antibody for 30 minutes, washed twice for 5 minutes in TBS, incubated for 30 minutes with RTU Vectastain Elite ABC Reagent (Vector Laboratories), washed in TBS stained with DAB Substrate kit (Vector Laboratories) according to the manufacturer's instructions, counterstained with hematoxylin and mounted in Vectashield hardest mounting medium (Vector Laboratories).

Animals: 6-12 week old C57BL/6 mice were purchased from the Jackson Laboratory and housed under pathogen-free conditions. The studies were approved by the Animal Care and Use Committee at the University of Pittsburgh. The design of the let-7d antagomir was adapted from Krutzfeldt et al, Nature (2005); 438, 685-689. The single-stranded RNA 5′-aacuaugcaaccuacuaccucu-3′ (SEQ ID NO: 7) was custom synthesized from Dharmacon, Lafayette, Colo. The sequence of the oligonucleotide was complementary to that of mmu-let-7d. All bases had 2′-O-methyl modifications, the first two bases and the last four bases had phoshorothioate linkages and a cholesterol molecule was conjugated at the 3′ end. In the shorter protocol, 4 mice were treated either with 10 mg/kg body weight let-7d antagomir administered intratracheally in 50 μl or an equal volume of saline. In the extended protocol, three groups with 2 mice in each group were administered 5 mg/kg of the antagomir. Group 1: Antagomir on days 1, 2, 3, 8, 9, 10 and sacrificed on day 11; group 2: antagomir on days 1, 2, 3, 8, 9, 10 and sacrificed on day 15; group 3: antagomir on days 1, 2, 3, 8, 9, 10, 15, 16, 17 and sacrificed on day 18. Two control mice were administered the same volume of saline in each of the three groups.

Masson's Trichrome staining: Sections were stained as per the established protocol Gomori G., American journal of clinical pathology (1950); 20, 661-664 by the University of Pittsburgh Transplantation Institute Core Facility.

Synthesis of antagomir: The single-stranded RNA 5′-aacuaugcaaccuacuaccucu-3′ was custom synthesized from Dharmacon, Lafayette, Colo. The sequence of the oligonucleotide was complementary to that of mmu-let-7d. All bases had 2′-O-methyl modifications, the first two bases and the last four bases had phoshorothioate linkages and a cholesterol molecule was conjugated at the 3′ end.

6.1.2 Results

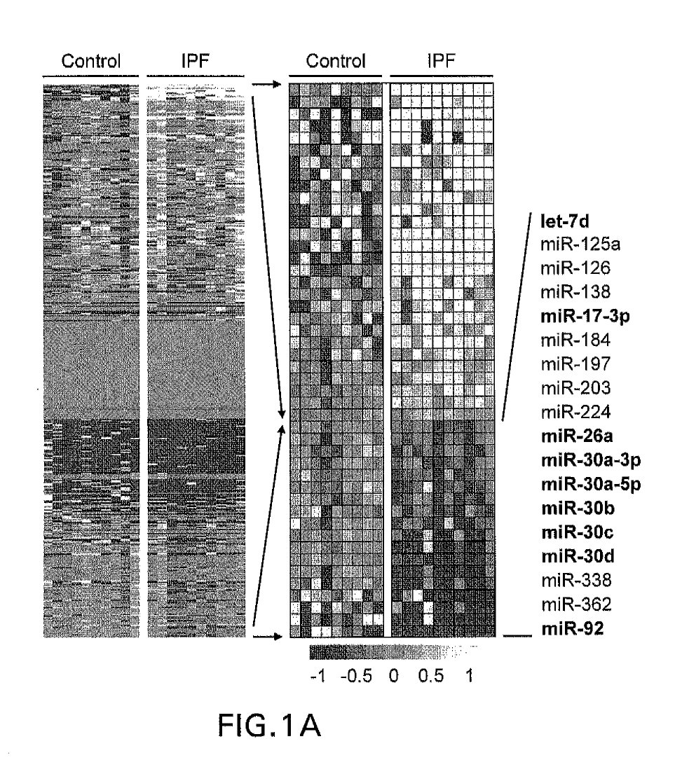

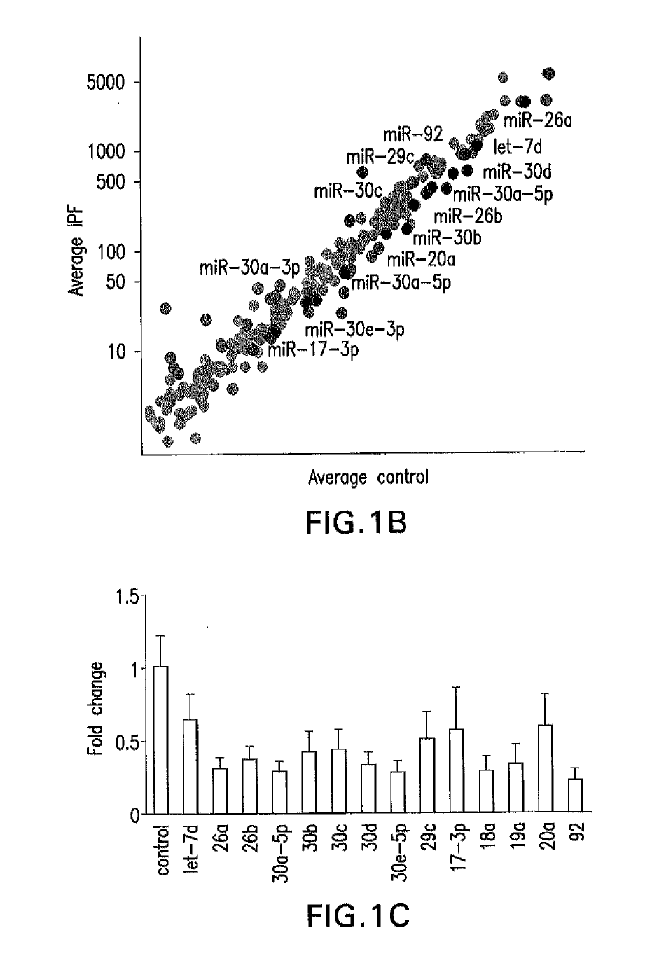

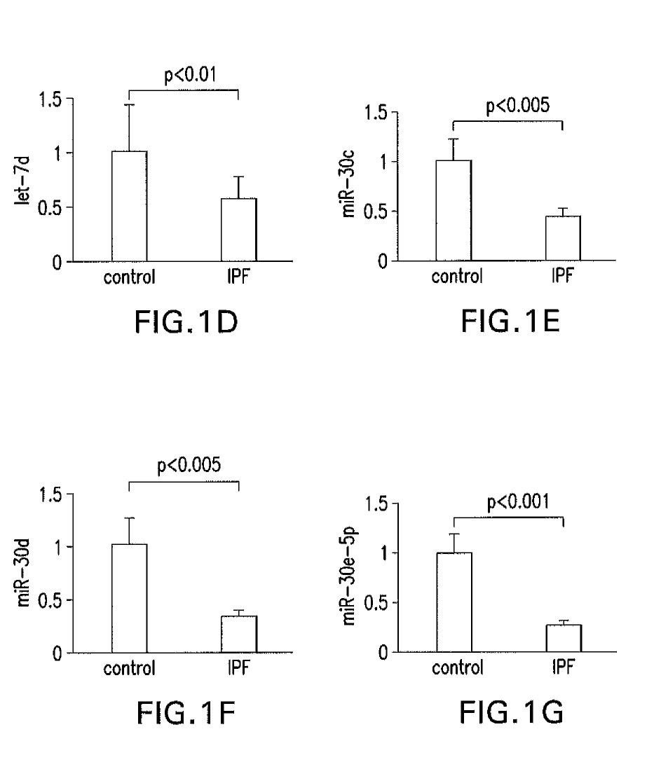

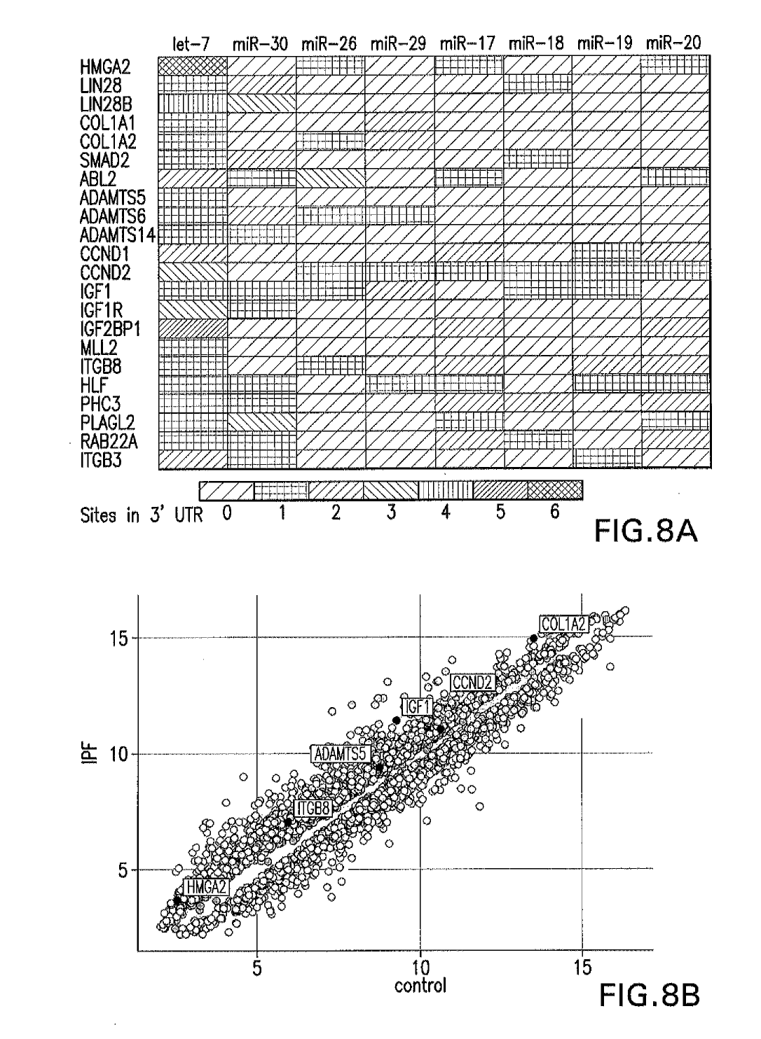

microRNAs are differentially expressed in IPF lungs. To determine differentially expressed microRNAs in IPF, total RNA of 10 IPF and 10 control lungs was hybridized on the Agilent microRNA microarrays. The complete microRNA microarray data has been deposited in the Gene Expression Omnibus (GSE13316) and is publicly available. 46 microRNAs were significantly differentially expressed in IPF lungs. Among the significantly decreased/down-regulated microRNAs in IPF lungs were let-7d, miR-26 family and several members of mir-30 family (FIG. 1A-B) which was also validated by qRT-PCR (FIG. 1C-G). Most of these microRNAs were differentially expressed during development and in various cancers. Analysis of microRNA target databases (Targetscan—http://www.targetscan.org, miRanda—http://www.microrna.org/ and PicTar—http://pictar.org) revealed that down-regulated microRNAs had overlapping targets—some of them increased in IPF lungs previously analyzed by gene expression microarrays (Konishi K. et al. American journal of respiratory and critical care medicine (2009); 180:167-175) (FIG. 8).

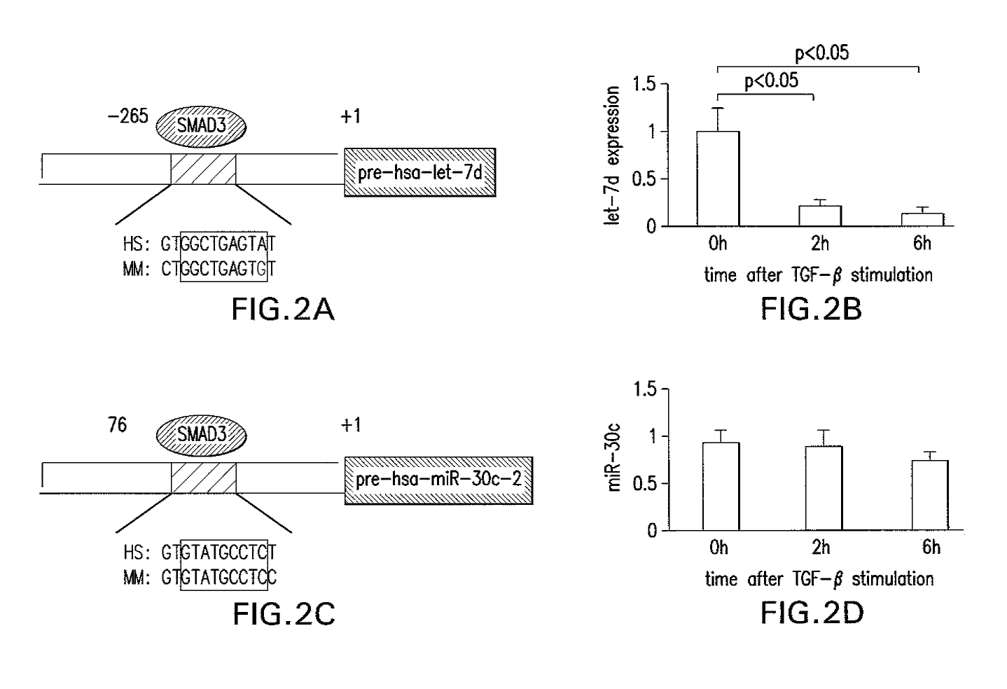

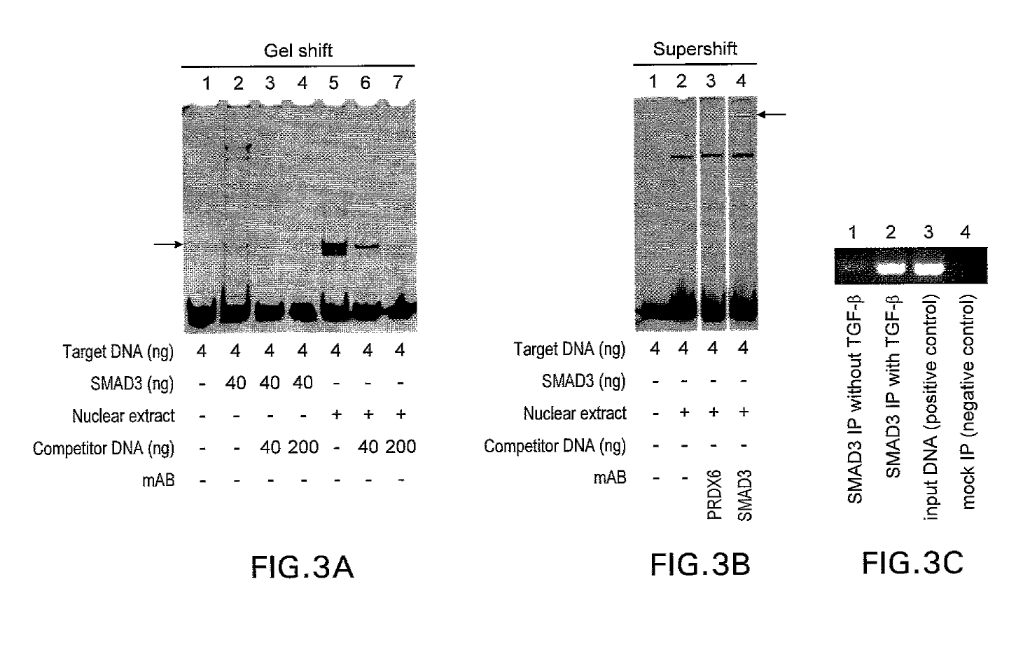

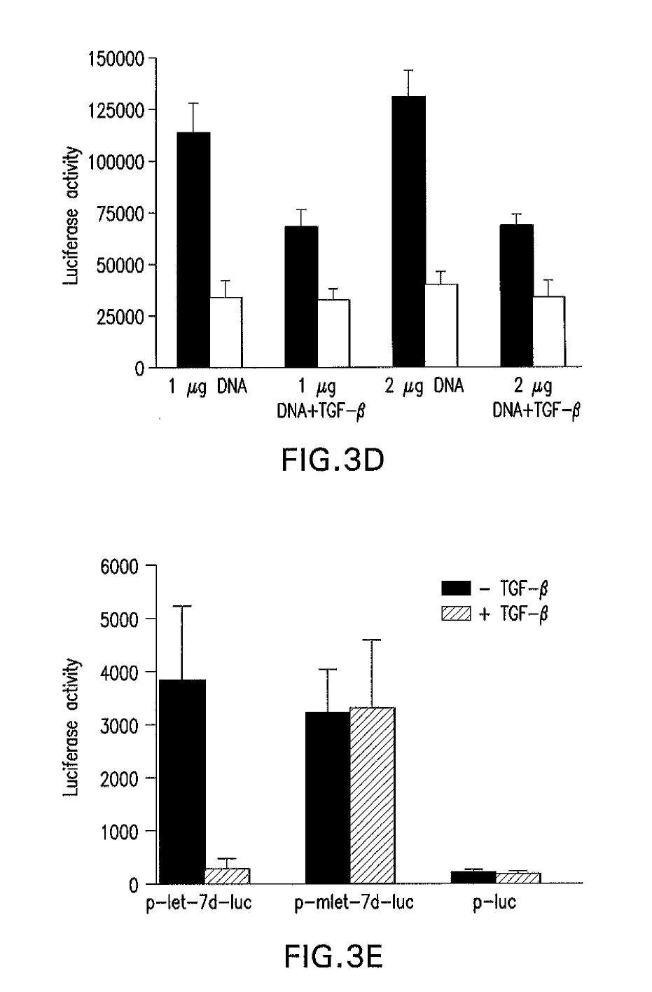

Let-7d expression is regulated by SMAD3 binding to its promoter. To determine whether any of the differentially regulated microRNAs was regulated by TGF-β, the FOOTER algorithm (Corcoran D. L., et al. Genome research (2005); 15:840-847 (2005) was used to locate potential SMAD binding sites in the putative promoter. SMAD binding sites were identified in the regions upstream of let-7d (FIG. 2A) and miR-30c-2 (FIG. 2C). To determine whether TGF-β indeed affected the expression of these microRNAs, A549 cells were stimulated with recombinant TGF-β. The expression of let-7d was significantly suppressed by TGF-β stimulation (p-value <0.005, 85% reduction) (FIG. 2B), while that of miR-30c remained unchanged (FIG. 2D). To confirm the computational prediction of SMAD3 binding to the putative promoter of let-7d, electrophoretic mobility shifty assay (EMSA) and chromatin immunoprecipitation (ChIP) were performed. Incubation of target DNA with either recombinant SMAD3 protein or nuclear extract of A549 cells revealed distinct bands representing the binding of SMAD3 to the let-7d promoter sequence (FIG. 3A). The intensity of these bands diminished in the presence of increasing concentrations of competitor DNA. The supershift band representing the DNA-protein-antibody complex was visible with SMAD3 antibody but not with the control peroxiredoxin 6 antibody confirming specificity of the reaction (FIG. 3B). SMAD3 ChIP revealed minimal binding of SMAD3 with the let-7d promoter with a dramatic increase after TGF-β stimulation (FIG. 3C). The promoter activity of the 5′ region of let-7d was further analyzed using a luciferase reporter assay (FIG. 3D-E). The 1687 base pair region (−1600 to +87) and an 8 basepair SMADs binding site deletion mutant were PCR amplified and cloned into the 5′ end of the luciferase gene. The reporter construct and empty vector controls were transfected into A459 cells with and without TGF-β activation and luciferase activity measured. The 1687 base pair region increased the average reporter activity by 25 fold. Luciferase activity was reduced to less than 30% in TGF-β treated cells as compared to untreated controls. This TGF-β mediated inhibition of luciferase activity was eliminated when the 8 base pair SMAD3 binding site deletion mutant was used (FIG. 3E). The presented results are representative of at least three experiments. Taken together, these results indicate that TGF-0 inhibits let-7d expression and that this inhibition is medicated through SMAD3 binding of the let-7d promoter.

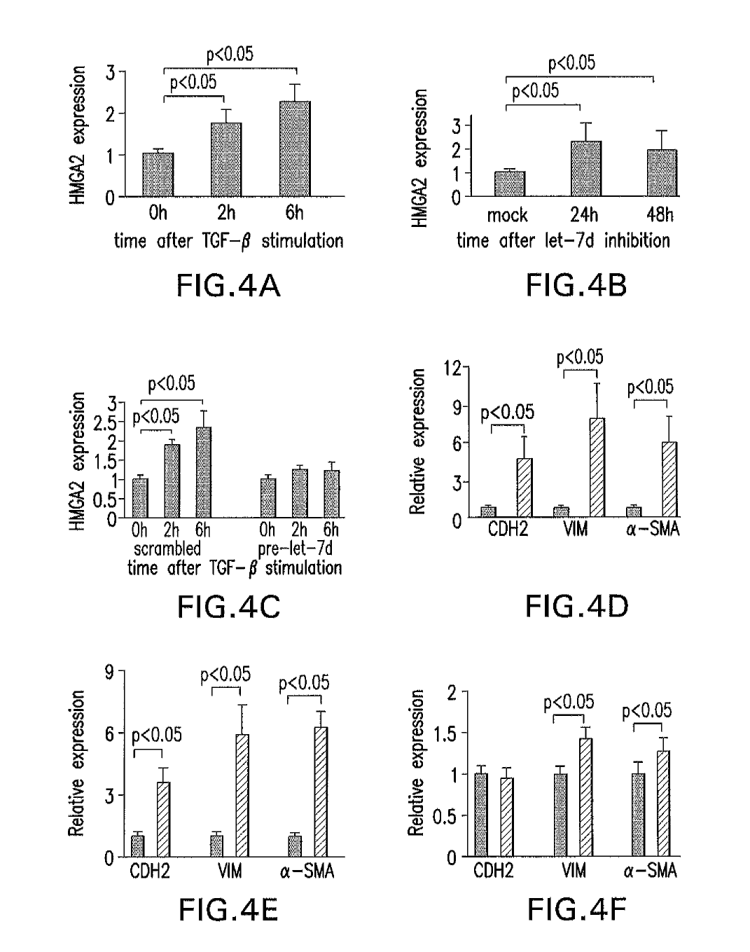

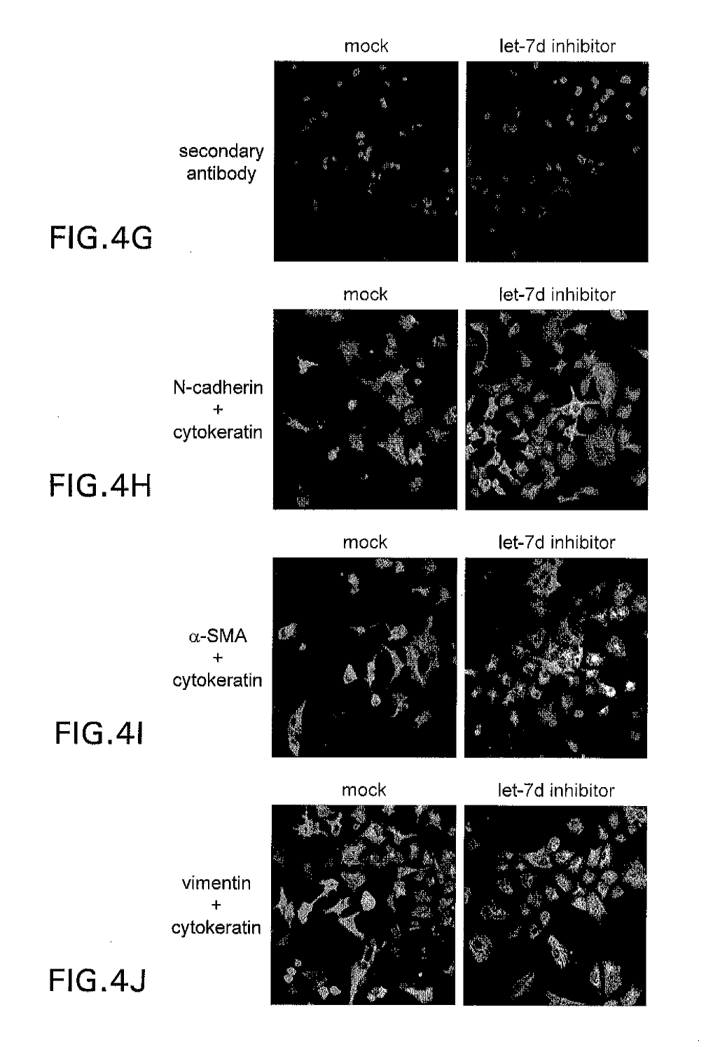

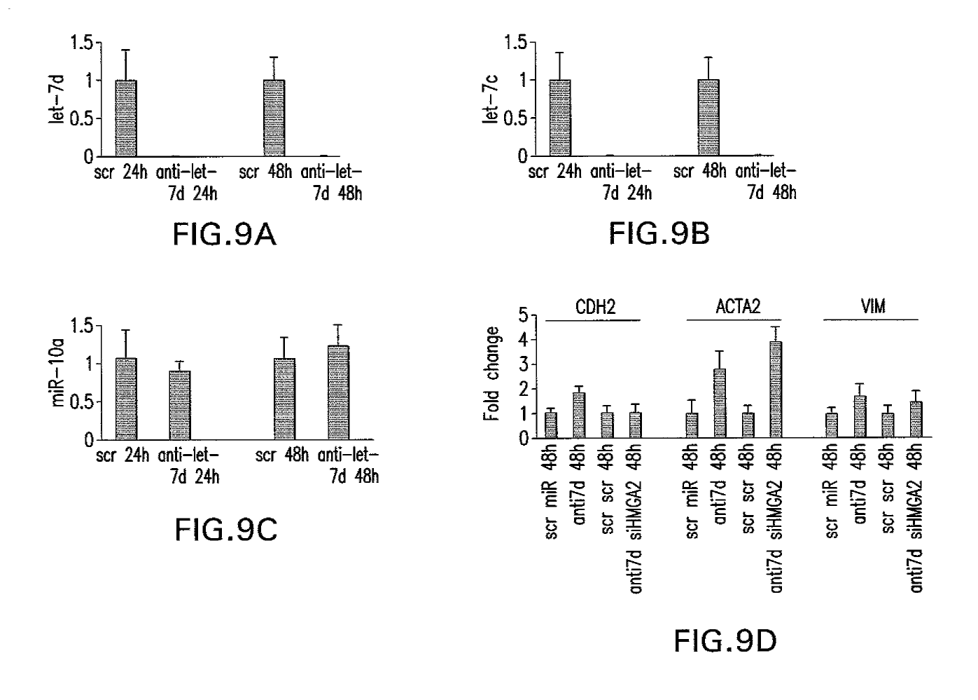

Inhibition of let-7d causes expression of mesenchymal markers in lung epithelial cell lines. To determine the potential roles of let-7d inhibition in IPF, the effects of let-7d on epithelial cell phenotype were studied Inhibition of let-7d induced a significant (p<0.05) and dramatic increase in expression of the mesenchymal markers N-cadherin (CDH2), vimentin (VIM) and alpha smooth muscle actin (ACTA2) in A549 cells (FIG. 4D), RLE-6TN cells (FIG. 4E) and primary bronchial epithelial cells (FIG. 4F). Inhibition of HMGA2 expression did not fully ablate this effect (FIG. 9D) suggesting that it was not completely mediated through HMGA2. Immunofluorescence confirmed the results at the protein level. A549 cells transfected with a let-7d inhibitor stained positive (red) for N-cadherin (CDH2), vimentin (VIM) and alpha-smooth muscle actin (ACTA2) 48 hours post-transfection, while mock transfected cells did not express positive staining (FIG. 4G-J).

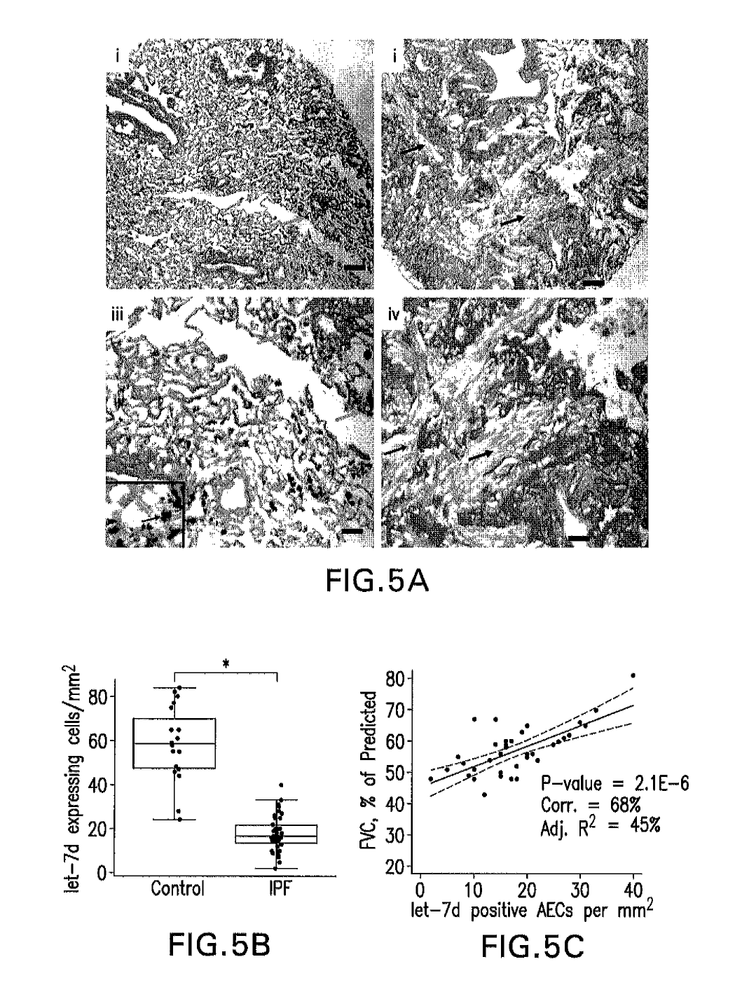

Let-7d is localized to alveolar epithelium in the normal lung and is significantly decreased in IPF alveolar epithelium. qRT-PCR was performed to confirm the previously described microarray analysis revealing that let-7d was down-regulated in IPF lungs (p-value <0.01) (FIG. 1D-G). To localize and quantify let-7d expression in the lungs, in situ hybridization on tissue microarrays containing 40 IPF tissues and 20 control lungs previously generated by Tzouvelekis A. et al., (Tzouvelekis A. et al., Am J Respir Crit Care Med (2007); 176:1108-1119), was performed. Control lungs exhibited abundant expression of let-7d in alveolar epithelial cells (FIG. 5A, panels i and iii). In contrast let-7d expression was almost absent in the alveolar epithelium within areas of fibrotic changes, or adjacent to fibroblastic foci in IPF lungs (FIG. 5A, panels ii and iv). The benefit of tissue arrays is that they allow the analysis of multiple lungs in parallel. Let-7d positive alveolar epithelial cells (AECs) in each tissue core (three in total for each patient) of diameter of 1.5 mm were counted. As seen in FIG. 5B, the number of let-7d positive cells/mm² was significantly lower (p<0.001) (17.9±7.9) in IPF patients compared to control lung samples where 57.2±17.6 AECs/mm² were positive for let-7d. Impressively the number of let-7d positive cells positively correlated with the forced vital capacity (FVC), a physiological indicator of disease progression (FIG. 5C).

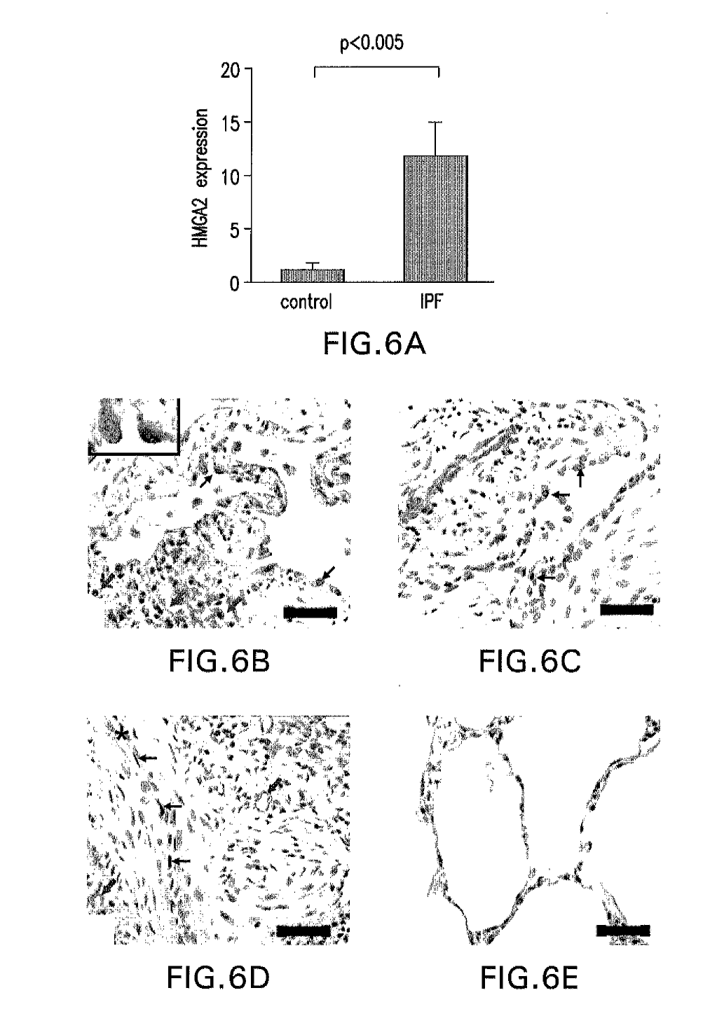

Decreased let-7d results in up-regulation of HMGA2 in vitro and in vivo. Since HMGA2 is a known target of let-7 (Mayr C., et al., Science (New York, N.Y.) (2007); 315:1576-1579) and is also a key regulator of EMT, its expression in IPF as a marker of let-7 down-regulation was studied. HMGA2 expression was increased in IPF lungs in previously published microarray data at Rosas I. O., et al., PLoS medicine (2008); 5:e93. qRT-PCR performed on the same samples used for miRNA microarrays confirmed the previous microarray data and revealed a 12-fold increase in HMGA2 (p<0.005) (FIG. 6A). Immunohistochemistry revealed expression of HMGA2 in alveolar epithelial cells and in some capillary endothelial cells of IPF lungs but not controls (FIG. 6B-E). To establish a potential relationship between the down-regulation of let-7d and concomitant up-regulation of HMGA2 in IPF lungs, whether let-7d was a regulator of TGF-β induced HMGA2 expression in lung epithelial cell lines was investigated. TGF-β stimulation caused a significant increase in HMGA2 (p<0.005) (FIG. 4A). Transfection of A549 cells with a let-7d inhibitor for 24 hours and 48 hours led to a significant increase in HMGA2 at 24 hours that remained elevated at 48 hours (FIG. 4B) an effect that was also observed in RLE-6TN cells. Over-expression of let-7d in RLE-6TN cells by transfection with pre-let-7d prevented TGF-β mediated induction of HMGA2 (FIG. 4C). These results demonstrate that let-7d is a regulator of HMGA2 expression in lung epithelial cell lines and that the increase in HMGA2 after TGF-β stimulation is in part dependent on inhibition of let-7d by TGF-13. The let-7d inhibitor is specific to the let-7 family of microRNAs (FIG. 9A-B) but does not affect the levels of unrelated microRNAs (FIG. 9C).

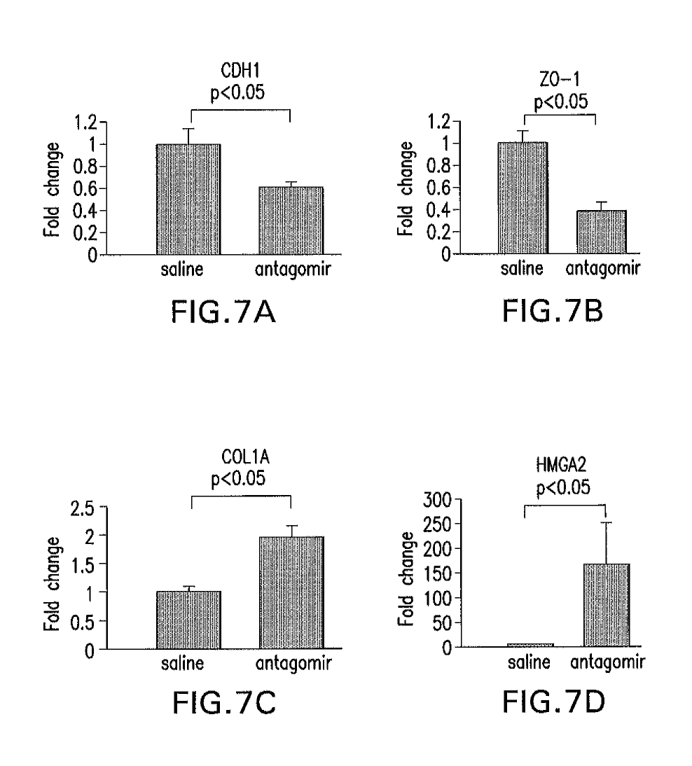

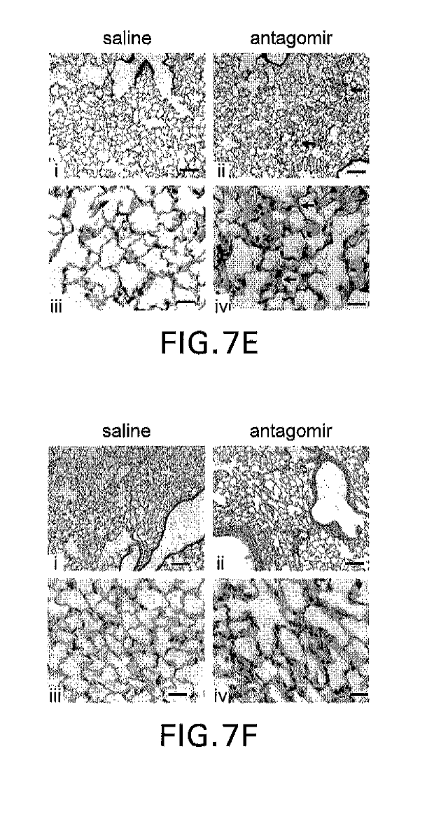

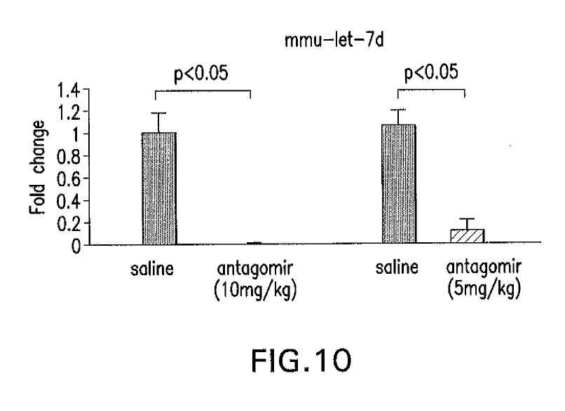

Inhibition of let-7d in vivo caused decreased CDH1 and ZO-1 expression, increased collagen, HMGA2 and ACTA2 expression, and thickening of alveolar septa. To determine the effects of let-7d inhibition in-vivo, the let-7d antagomir designed as described at Krutzfeldt J. et al., Nature (2005); 438:685-689, was administered intratracheally at a dose of 10 mg/kg body weight into the lungs of 4 mice on three consecutive days and sacrificed the mice on the fourth day. Control mice were administered an equal volume of saline. The intratracheal administration of the let-7d antagomir caused a complete knockdown of let-7d in the lung (FIG. 10). Let-7d inhibition caused a significant decrease in the expression of the epithelial markers CDH1 and ZO-1 (FIG. 7A-B) and a significant increase in COL1A1 and HMGA2 expression in the lung (FIG. 7C-D). At this dose the mice appeared sick and the lungs were grossly hemorrhagic and it was impossible to maintain them longer. To better observe the long term effects of let-7d inhibition, the dose was reduced to 5 mg/kg body weight and the animals were treated for 18 days. At this dose the mice did not demonstrate any discomfort, but a significant decrease in let-7d in the lung was detected (FIG. 10). Masson's trichrome stain of formalin fixed mouse lung demonstrated increased thickening of alveolar septa and increased positive stain (blue) indicative of the presence of collagen in let-7d antagomir-treated lungs (FIG. 7E). Immunohistochemical staining for ACTA2 revealed enhanced immunoreactive protein in alveolar walls in antagomir-treated mice lungs that was not observed in saline treated mice (FIG. 7F). Taken together, these results suggest that in vivo inhibition of let-7 in the lung causes changes consistent with changes in epithelial cell phenotype (decrease in CDH1 and ZO1) and consistent with early fibrosis (thickened alveolar septa, increased collagen and ACTA2).

6.1.3 Discussion

This study explored the expression, regulation and potential role of microRNAs in IPF. Previous studies have demonstrated that many microRNAs are both transcribed by RNA polymerase II (Lee Y., et al., The EMBO journal (2004); 23:4051-4060)) and share a similar sequence conservation pattern in the immediate upstream region as protein coding genes (Mahony S., et al., Genome biology (2007); 8:R84 (2007)). These observations suggest that microRNAs may share similar transcriptional regulatory mechanisms as other RNA polymerase II transcribed genes. Since SMAD transcription factors play a significant role in the signaling pathway of the key profibrotic cytokine TGF-β, SMAD binding sites were searched for in the promoters of the microRNAs that were significantly down-regulated in IPF. It was discovered that the promoter of one of the down-regulated microRNAs, let-7d, contained an SMAD binding site and confirmed its binding and responsiveness to TGF-β. Among the down-regulated microRNAs in IPF lungs, let-7d was the focal point of this study because of its regulation by TGF-β and its potential role in mechanisms relevant to IPF. It was discovered that let-7d was directly transcriptionally inhibited by the key profibrotic cytokine TGF-β. In-vitro inhibition of let-7d induced an increase in mesenchymal markers consistent with EMT in lung epithelial cell lines and in-vivo inhibition of let-7d in mouse lungs caused alveolar septal thickening, decreased expression of epithelial markers and increase in collagen and ACTA2 consistent with early fibrotic changes. In IPF lungs let-7d expression was drastically diminished, while HMGA2 expression was increased in alveolar epithelial cells. Increased expression of HMGA2 was induced by let-7d inhibition in vitro and TGF-β induced expression of HMGA2 was inhibited by let-7d expression suggesting that the concomitant decrease in let-7d and increase in HMGA2 in human lungs was associated. Taken together, these findings suggest that let-7d inhibition is a key regulatory event in the dramatic phenotypic changes that happen in the alveolar epithelium in IPF (Selman M. and Pardo A. Proceedings of the American Thoracic Society (2006); 3:364-372).

The let-7 family of microRNAs was one of the first to be discovered (Reinhart B. J. et al., Nature (2000); 403:901-906) and most extensively studied, however this is the first instance in which a member of let-7 family is implicated in a non-tumor disease. Let-7 family of microRNAs are temporally regulated RNAs which coordinate developmental timing (Reinhart B. J. et al., Nature (2000); 403:901-906). Their importance is reinforced by the fact that their sequence and temporal expression pattern are conserved in a variety of organisms (Pasquinelli A. E., et al., Nature (2000); 408:86-89). Apart from having a crucial role in development, the let-7 family of microRNAs also acts as a tumor suppressor. They have many complementary sites in the 3′ UTR of the RAS oncogenes (Johnson S. M., et al., Cell (2005); 120:635-647) and are down-regulated in a wide variety of cancers possibly due to their location at fragile sites (Calin G. A., et al., Proceedings of the National Academy of Sciences of the United States of America (2004); 101:2999-3004 (2004)). The let-7 microRNAs also represses several genes involved in the cell cycle (Johnson C. D., et al., Cancer research (2007); 67:7713-7722). In a recent study, Shell et al. conducted a study on the panel of NCI60 human tumor cell lines and concluded that let-7 is a good candidate to define the “epithelial” gene signature (Shell S. et al., Proceedings of the National Academy of Sciences of the United States of America (2007); 104:11400-11405) in agreement with these results that suggest that loss of let-7 may cause loss of epithelial characteristics. Mayr et al. demonstrated that let-7 was a negative regulator of HMGA2, a structural transcriptional regulator overexpressed in early embryonic development, some benign tumors and lung cancer (Mayr C. et al., Science (New York, N.Y.) (2007); 315:1576-1579). This is especially important because patients with IPF exhibit an increased risk for lung cancer even after adjusting by smoking, although the pathogenic mechanisms are largely unknown (Hubbard R. et al., American journal of respiratory and critical care medicine (2000); 161:5-8). Interestingly, HMGA2 confers a growth advantage to fibroblasts as evident by the retarded growth in hmga2-deficient mouse embryonic fibroblasts compared to wild-type fibroblasts (Zhou X. et al., Nature (1995); 376:771-774) and is also a mediator TGF-β induced EMT (Thuault S., et al., The Journal of cell biology (2006); 174:175-183), thus its overexpression may have pro-fibrotic effects in the lung. The HMGA group of proteins also promote and maintain proliferative arrest by accumulating on chromatin in senescent cells (Narita M. et al., Cell (2006); 126:503-514). The findings of decreased let-7d and increased HMGA2 in the lung alveolar epithelium are indicative of the extent of which alveolar epithelial cells in IPF are different fro normal epithelial cells and may also provide an explanation for the increases incidence of cancer in IPF patients. While a dramatic decrease in let-7d expression accompanied by an increased in HMGA2 in human IPF lungs was found, and it was demonstrated that inhibition of let-7d leads to EMT in vitro and to early fibrotic changes in vivo, there is no direct evidence that this effect is directly mediated through HMGA2. A more realistic interpretation at this stage would be that in the lung, down-regulation of let-7d leads to up-regulation of HMGA2 as well as other fibrosis relevant targets of let-7 such as RAS, IGF1, IGF1R (FIG. 8) and thus lead to the profound and sustained changes in cellular phenotype that were indeed observe in IPF. The fact that let-7 inhibition is sufficient to induce EMT in vitro and expression of mesenchymal markers as well thickening of alveolar septum indicative of early fibrosis in vivo also suggests an effect mediated through modification of the expression of multiple fibrosis relevant genes and not just a single gene. EMT is a physiologic phenomenon seen during embryogenesis, organ development and wound healing. Pathological examples of EMT are seen in aberrant tissue repair resulting in fibrosis and tumor progression. TGF-β is the main inducer of EMT (Nawshad A. et al., Cells, tissues, organs (2005); 179:11-23). A transcriptome screen to study the initial phase of TGF-β-induced EMT revealed that among the signaling modules almost 4,000 genes were differentially expressed (Zavadil J. et al., Proceedings of the National Academy of Sciences of the United States of America (2001); 98:6686-6691) suggesting extensive regulation of EMT at the transcriptional level. The data described herein as well as a few recent studies on the miR-200 family (Burk U. et al., EMBO reports (2008); 9:582-589; Korpal M., et al., The Journal of biological chemistry (2008); 283:14910-14914; Park S. M. et al., Genes & development (2008); 22:894-907; Gregory P. A. et al., Nature cell biology (2008); 10:593-601) have identified the role of microRNAs in EMT demonstrating additional regulation at the post-transcriptional level. The miR-200 family prevents EMT by targeting the transcription factors ZEB1 and SIP1 that repress E-cadherin, an epithelial cell marker (Burk U. et al., EMBO reports (2008); 9:582-589; Korpal M., et al., The Journal of biological chemistry (2008); 283:14910-14914; Park S. M. et al., Genes & development (2008); 22:894-907; Gregory P. A. et al., Nature cell biology (2008); 10:593-601). Similar to let-7d, TGF-β stimulation also decreased expression of the miR-200 family. Overexpression of the miR-200 family could not completely block EMT (Korpal M., et al., The Journal of biological chemistry (2008); 283:14910-14914) suggesting that let-7d and miR-200 family may be acting synergistically possibly with a few other microRNAs in preventing EMT. While the current study does not directly overlap the putative pathway of miR-200 family, there is no doubt that there may be multiple signaling cascades likely to be at work in a complex cellular transition such as EMT. Additionally, it is possible that in different organs or during different embryonic stages different microRNAs may have the role of maintaining epithelial cell phenotype. In this context, it is important to note that while the study described herein focuses on let-7d, the downstream effects of let-7d overexpression or inhibition are probably common to all members of the let-7 family of microRNAs. While the inhibitory effects described herein do not go through microRNAs that are not members of the let-7 family, it is highly unlikely that this effect will be mediated only through let-7d and not through other members of the family.

This study demonstrates the influence of a growth factor on the expression of a microRNA through direct effect on its promoter. Growth factors play an important role in the regulation of cell proliferation and differentiation. The response of cells to growth factors is often in the form of alteration in gene expression. Growth factors, which are regulators of gene expression, alter the levels of another set of gene expression regulators (microRNAs) and add to the complexity of the various interacting intracellular pathways. This may also suggest that regulation of microRNA expression may be more dynamic then previously thought. The recent genome scale analysis of microRNA promoters and evidence for binding by stem cell transcription factors as well as RNA polymerase II also supports this notion (Corcoran D. L. et al., PLoS ONE (2009); 4:e5279; Marson A. et al., Cell (2008); 134:521-533). So far changes in let-7 microRNAs expression levels have been mostly attributed to their localization to fragile sites (Calin G. A. et al., Proceedings of the National Academy of Sciences of the United States of America (2004); 101:2999-3004), to post-transcriptional modification (Thomson J. M. et al., Genes & development (2006); 20:2202-2207), or to epigenetic changes (Brueckner B. et al., Cancer research (2007); 67:1419-1423). The discovery that let-7d is directly inhibited by TGF-β adds a new potential mechanism for regulation of microRNAs and may have important therapeutic implication as inhibitors of TGF-β signaling and activation are currently evaluated for cancer and fibrosis. Furthermore, the suggestion that some of TGF-β effects on epithelial cells may be mediated through microRNAs may have significant implications in better understanding the profound and sustained effects of TGF-β on cell and organ phenotype.

In summary, it was demonstrate that IPF lungs are different from control lungs in their microRNA repertoire. Let-7d, a microRNA significantly down-regulated in IPF, was negatively transcriptionally regulated by TGF-13. Furthermore, inhibition of let-7d alone is sufficient to cause EMT in A549 cells and RLE-6TN cells. Concomitant with let-7d down-regulation in IPF lungs, significant increases in lung expression of HMGA2 was found, which suggests that let-7 inhibition of HMGA2 is released in IPF lungs. Finally, it was demonstrated that let-7 inhibition in vivo in the lung may cause changes in the lung alveolar epithelium with increases in mesenchymal markers, decreases in epithelial markers and thickening of alveolar septa. The discovery of the down-regulation of let-7d in IPF, its regulation by TGF-β and its potential role in EMT and fibrosis may have important implications in the understanding of the molecular mechanisms underlying IPF as well lead to the development of new therapeutic interventions in this devastating and incurable disease.

### 6.2. Example 2

### Expression of miR-30 Family in IPF

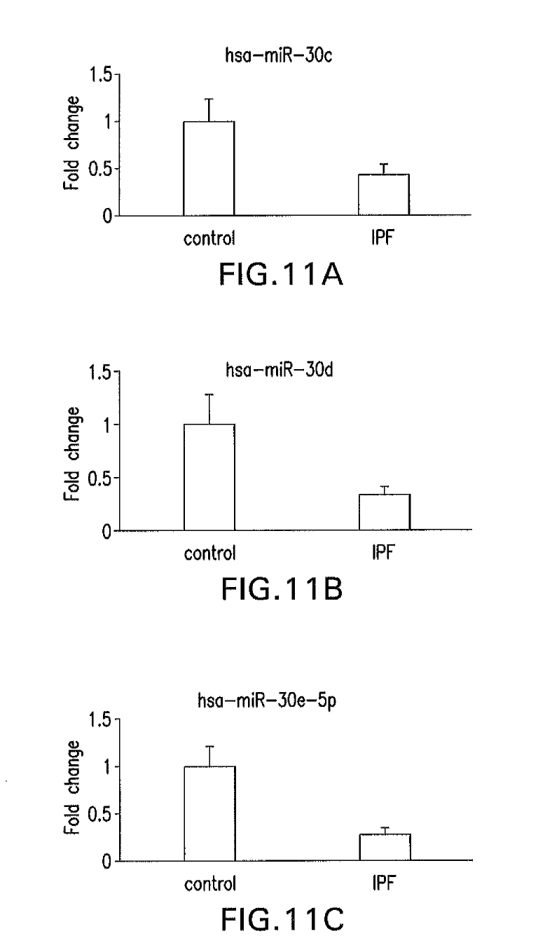

Microarray analysis of 10 IPF lung tissues and 10 control lungs showed down-regulation of the miR-30 family of microRNAs. Real-time PCR was performed in order to confirm whether down-regulation of these microRNAs occurs in IPF. FIGS. 11A-C show the results of these PCR studies, and show decreased levels of hsa-miR-30c (FIG. 11A), hsa-miR-30d (FIG. 11B) and hsa-miR-30e-5p (FIG. 11C).

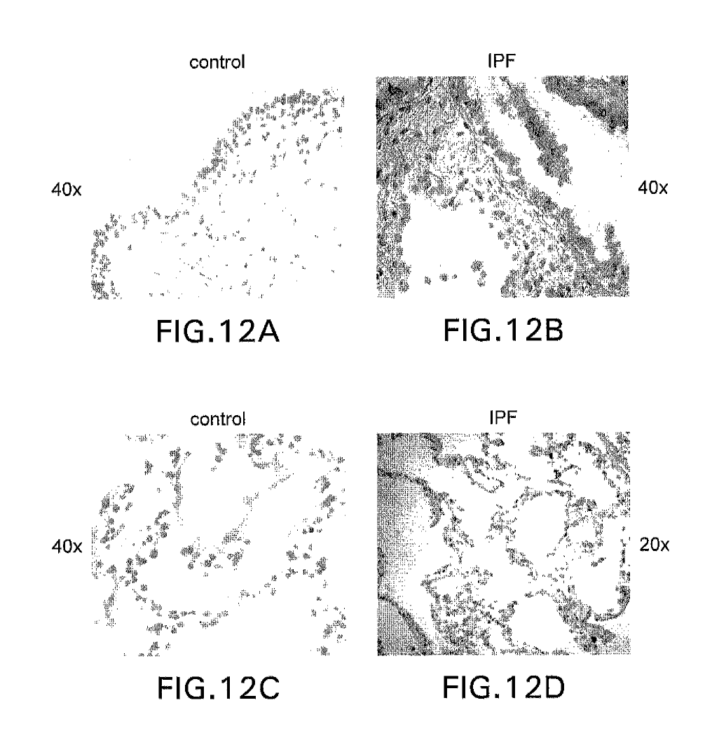

To localize miR-30 in the lungs, in situ hybridization was performed for miR-30e on tissue sections of IPF and control lungs. As shown in FIG. 12A-D, increased expression of miR-30e was observed in the control lungs, where the microRNA appeared to be localized to the alveolar epithelial cells (FIG. 12B).

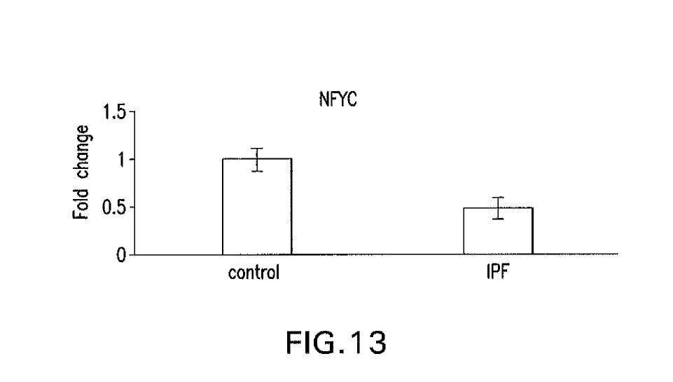

Interestingly, miR-30c and miR-30e are in the intron of NFYC, a suppressor of SMAD2 and SMAD3 transactivating activity. NFYC was also down-regulated in IPF as evident by the qRT-PCR results shown in FIG. 13. Generally, intronic microRNAs are transcribed with their host gene.

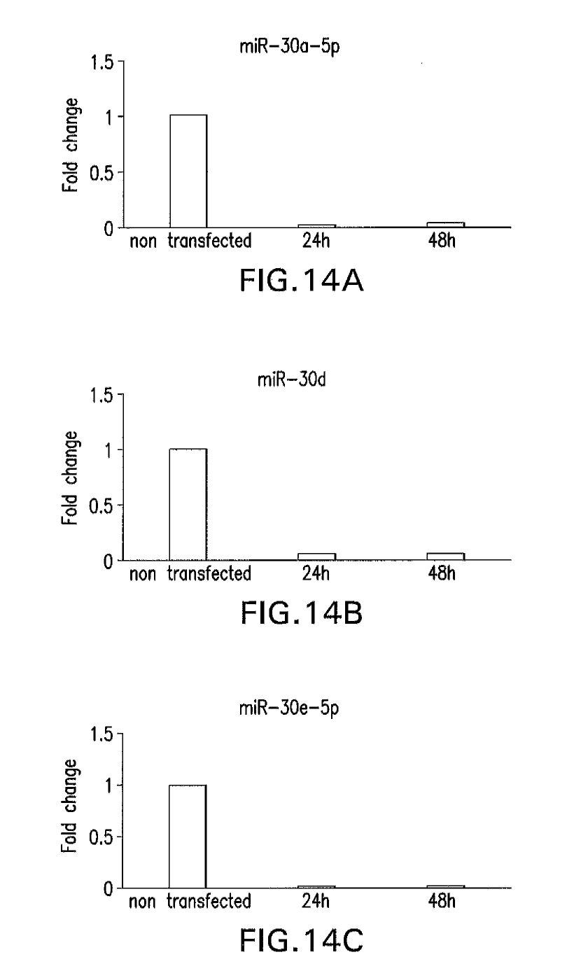

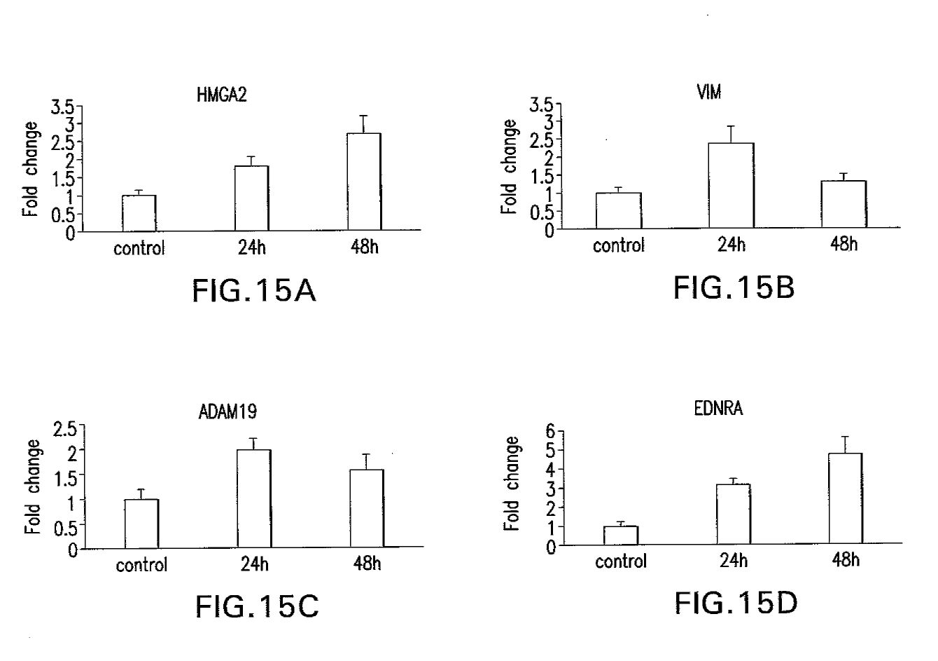

Functional analyses were performed in order to determine biologically relevant targets of the miR-30 family. To achieve this, an LNA/DNA test oligonucleotide was designed which was intended to inhibit miR-30a-5p, miR-30d and miR-30e-5p (but not miR-30b or miR-30c) because 30a-5p, 30d and 30e4-5p microRNAs share a very similar sequence which miR-30b and miR-30c lack. This test oligonucleotide (5′ CTTCCAGTCGGGGATGTTTACA 3′ (SEQ ID NO: 8), where underlined bases are LNA bases and the rest of them are DNA bases, see Mott J. L. et al., Oncogene (2007);26(42):6133-40) was transfected into A549 lung alveolar epithelial cells and the expression levels of 30a-5p, 30d and 30e4-5p microRNAs was assessed by qRT-PCR at 24 hours and 48 hours post-transfection. The results, demonstrating effective inhibition, by the oligonucleotide, of expression of these microRNAs, are shown in FIG. 14A-C. The levels of certain mRNAs which are targets of miR-30 and are relevant in IPF (e.g. HMGA2, vimentin, ADAM19 and EDNRA (endothelin receptor A)) were also measured in the oligonucleotide transfected cells, and the results are shown in FIG. 15A-D. The levels of HMGA2 and EDNRA mRNAs were found to increase during the 48 hour period (FIGS. 15A and D, respectively), whereas the levels of VIM and ADAM19 mRNAs were found to decrease (FIGS. 15B and C, respectively).

Among these results, the increase in EDNRA mRNA expression is of particular interest in view of a report by Jain et al. regarding a possible role of EDNRA in epithelial mesenchymal transition (EMT) (Jain R. et al., Am J Respir Cell Mol Biol. (2007); 37: 38-47). These authors showed that alveolar epithelial cells produce endothelin-1, the ligand for EDNRA. TGF-β increases the production of endothelin-1 and vice versa. Jain et al. also demonstrated that endothelin-1-induced EMT is mediated by TGF-β (Jain R. et al., Am J Respir Cell Mol Biol. (2007); 37: 38-47).

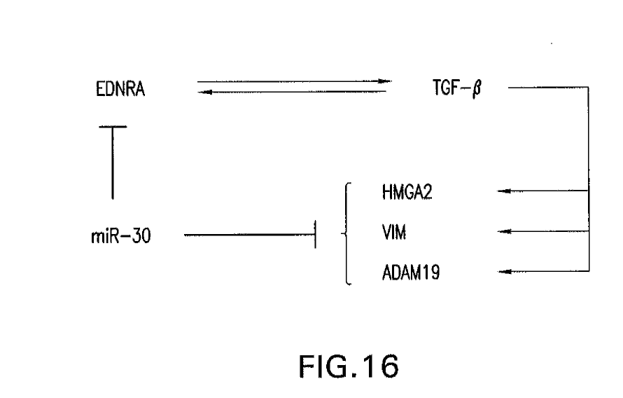

In view of the results discussed above, in IPF, miR-30 may be involved in EMT by the following two mechanisms:
(i) Down-regulation of miR-30 in IPF leads to overexpression of its target EDNRA which in turn increases the expression of TGF-β. The downstream effect of increase in TGF-β is the corresponding increase in HMGA2, vimentin and ADAM19.
(ii) However, HMGA2, vimentin and ADAM19 are also direct targets of miR-30.
   A diagram of this proposed mechanism is shown in FIG. 16.

### 6.3. Example 3

### Expression of Other MicroRNAs in IPF

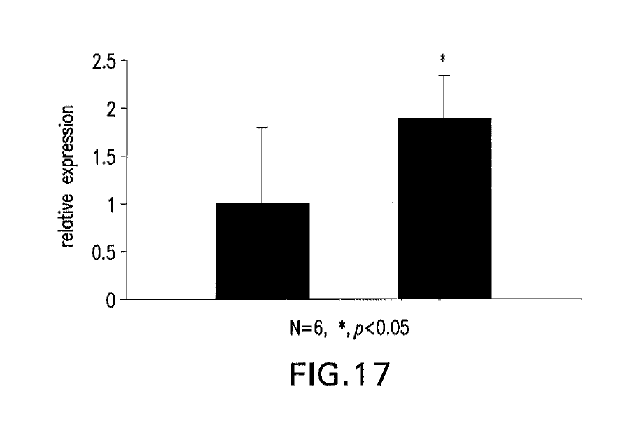

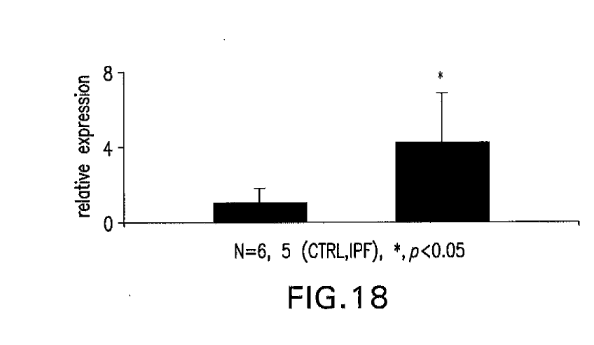

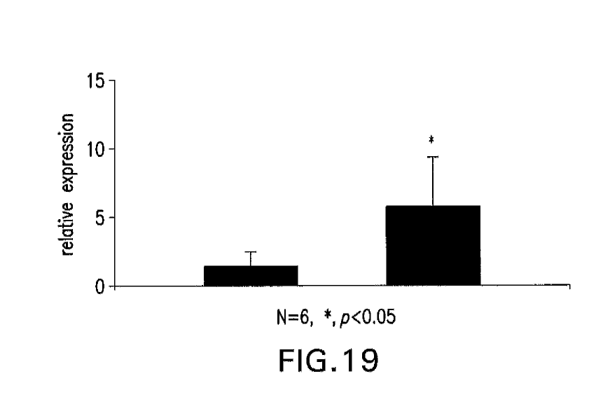

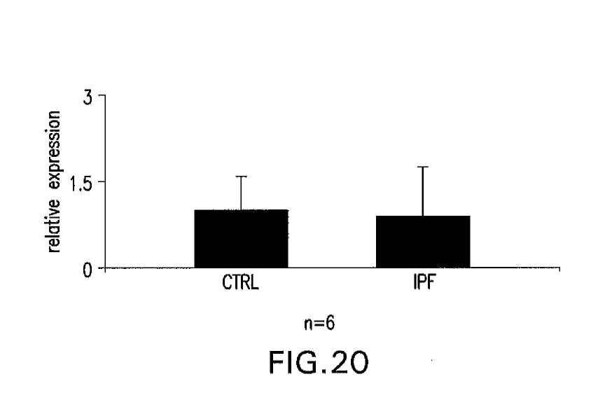

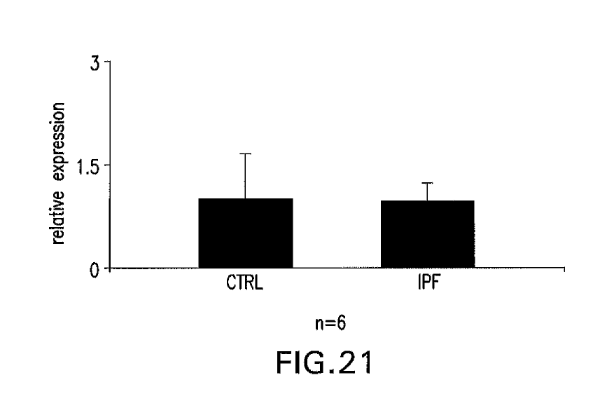

Real-time PCR analysis was performed to determine the levels of other microRNAs in IPF as compared to normal lung tissue. As shown in FIGS. 17-21, the levels of hsa-mir-154, hsa-mir-205 and hsa-mir-31 were increased in IPF tissue, whereas the levels of hsa-mir-324-3p and hsa-mir-155 were the same or slightly increased or decreased, respectively, relative to normal levels (FIGS. 20 and 21, respectively).

### 6.4. Example 4

Methods: microRNA profiling was carried out on 10 IPF lungs and 10 control lungs using the Agilent Human miRNA Microarray. These microarrays have an 8×15K design with 470 microRNAs based on Release 9.1 of Sanger miRBASE. The microarray results were verified by qRT-PCR.

Results: Out of 470 microRNAs on the array 57 were differentially regulated (FDR=0.1). Among the down-regulated microRNAs in IPF were let-7d, miR-26, miR-29c and multiple members of the mir-30 family. These decreases were verified by RT-PCR. Some predicted targets of the mir-30 family such as COL1, HMGA2 are overexpressed (p<0.05) in IPF lungs. Interestingly, miR-30c and miR-30e are located in the intron of NFYC, a repressor of the key TGFβ1 transcription factor SMAD3. NFYC mRNA was also significantly decreased (p<0.05) in IPF lungs.

### 6.5. Example 5

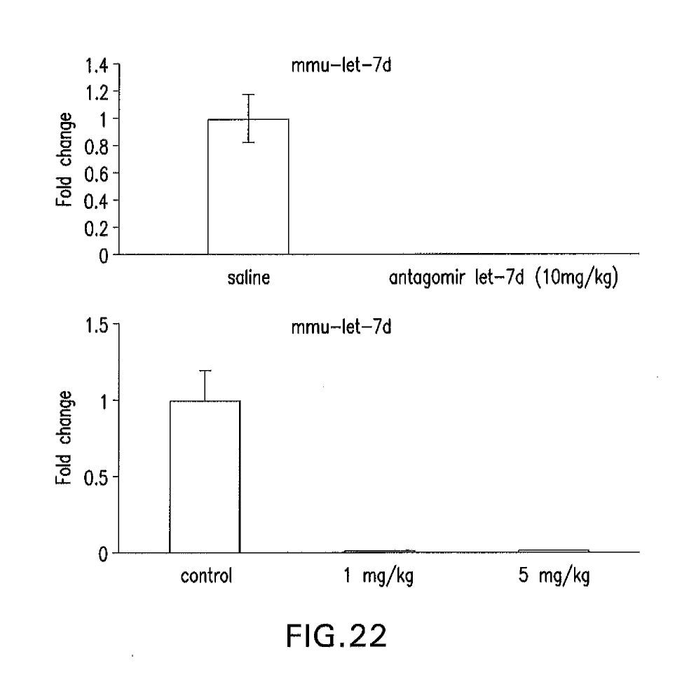

Antagomir oligonucleotide, at various concentrations, was injected intratracheally into mice. The mice were sacrificed at day 4, and the levels of let-7d were measured. The results, shown in FIG. 22, show that Antagomir even at the lowest concentration, 1 mg/kg, was effective at suppressing expression of let-7d in mouse lung.

Various publications are cited herein, the contents of which are hereby incorporated by reference in their entireties.

1. A method of diagnosing idiopathic pulmonary fibrosis in a subject comprising measuring, in a lung tissue sample of the subject, the level of one or more microRNA, wherein a decrease in the level of one or more microRNA selected from the group consisting of let-7d, mir-30d, mir-30c, mir-30e-5p, miR-26a, miR-26b, miR-29c and mir-17-˜92 cluster, relative to one or more control value, indicates a diagnosis of idiopathic pulmonary fibrosis in the subject.

2. A method of diagnosing idiopathic pulmonary fibrosis in a subject comprising measuring, in a lung tissue sample of the subject, the level of one or more microRNA, wherein an increase in the level of one or more microRNA selected from the group consisting of hsa-mir-154, hsa-mir-205, and hsa-mir31, relative to one or more control value, indicates a diagnosis of idiopathic pulmonary fibrosis in the subject.

3. The method of claim 1, further comprising administering one or more pulmonary function test to the subject, in order to confirm the diagnosis of idiopathic pulmonary fibrosis.

4. The method of claim 1, wherein the level of one or more microRNA is measured by a hybridization-based method.

5. The method of claim 1, wherein the level of one or more microRNA is measured by an amplification-based method.

6. A method of treating idiopathic pulmonary fibrosis in a subject in need of such treatment comprising administering, to the subject, an agent which increases the level of one or more microRNA selected from the group consisting of let-7d, mir-30d, mir-30c, mir-30e-5p, miR-26a, miR-26b, miR-29c and mir-17-˜92 cluster in lung tissue of the subject.

7. A method of treating idiopathic pulmonary fibrosis in a subject in need of such treatment comprising administering, to the subject, an agent which decreases the level of one or more microRNA selected from the group consisting of hsa-mir-154, hsa-mir-205, and hsa-mir31, in lung tissue of the subject.

8. A kit to be used in diagnosing idiopathic pulmonary fibrosis comprising means of detecting a change in the level of one or more microRNA species associated with idiopathic pulmonary fibrosis selected from the group consisting of let-7d, mir-30d, mir-30c, mir-30e-5p, miR-26a, mir-26b, miR-29c, mir-17-˜92 cluster, hsa-mir-154, hsa-mir-205, and hsa-mir-31, wherein said one or more microRNA species associated with idiopathic pulmonary fibrosis constitutes at least about 20 percent of the total number of RNA species represented in the kit.

9. An isolated nucleic acid having the sequence as set forth in SEQ ID NO: 7, where all bases are 2′-OMe-modified, the first two and last four bases have phosphorothioate linkages, and a cholesterol molecule is attached at the 3′ end.
